(19) United States
(12) Patent Application Publication
(10) Pub. No.: US 2002/0081617 A1
(43) Pub. Date: Jun. 27, 2002

(54) Fluorescence and FRET based assays for biomolecules on beads

(76) Inventors: Tione Buranda, Albuquerque, NM (US); Victor H. Perez-Luna, Naperville, IL (US); Gabriel P. Lopez, Albuquerque, NM (US); Peter Simons, Albuquerque, NM (US); Jinman Huang, Albuquerque, NM (US); Larry A. Sklar, Albuquerque, NM (US)

Correspondence Address: Ajay A. Jagtiani, Jagtiani + Guttag, Democracy Square Business Center, 10379-B Democracy Lane, Fairfax, VA, 22030, US

(21) Appl. No.: 09/985,873

(22) Filed: Nov. 6, 2001

Related U.S. Application Data
(60) Provisional application No. 60/246,564, filed on Nov. 8, 2000.

(51) Int. Cl. C12Q 1/68; G01N 33/53; C12M 1/34

(52) U.S. Cl. 435/6; 435/287.2; 435/7.1

(57) ABSTRACT

The invention provides a sensing device comprising: a vessel; a plurality of sensor beads located within the vessel to form interstitial spaces therethrough; and a plurality of biomolecules bound to at least at portion of the plurality of beads, each of the biomolecules having a fluorescent tag. The invention also provides a method for detecting the binding of two biomolecules comprising the following steps: providing a plurality of first biomolecules, each of the first biomolecules having a first fluorescent tag, each of the first biomolecules being bound to a respective substrate of a plurality of substrate; providing a plurality of second biomolecules, each of the second biomolecules having a second fluorescent tag, binding at least portion of the second biomolecules to at least a portion of the first biomolecules to form complexes, wherein the plurality of first biomolecules and the plurality of second biomolecules prior to the binding step have a pre-complexing total fluorescence and wherein the complexes and free second biomolecules after the binding step have a post-complexing total fluorescence; and detecting any difference between the pre-complexing total fluorescence and the post-complexing total fluorescence. A sensing device comprising a suspension of a plurality of sensor beads; and a plurality of biomolecules bound to at least a portion of the plurality of beads, each of the biomolecules having a fluorescent tag is also provided.

### CROSS-REFERENCE TO RELATED APPLICATIONS

This application makes reference to the following pending U.S. Patent Application. The application is U.S. App. No. 60/246,564, entitled “Bead-based Assay for Epitope Tags and Porous Affinity Sensor with Fluorescence Detection,” filed Nov. 8, 2000. The entire disclosure and contents of the above application is hereby incorporated by reference.

### GOVERNMENT INTEREST STATEMENT

This invention is made with government support under ONR grant #N00014-95-1-1312 with the Department of Defense through the Multidisciplinary Research Program of the University Research Initiative (Office of Naval Research), NSF #MCB-9907611 with the National Science Foundation, and NIH-BECON (GM60799-02). The government may have certain rights in this invention.

### BACKGROUND OF THE INVENTION

1. Field of the Invention

The present invention relates generally to fluorescence based bead binding assays, and more particularly to assays utilizing fluorescence resonance energy transfer (FRET) as the mode of detection.

2. Description of the Prior Art

Standard microplate based sandwich immunoassays such as ELISAs are time consuming and involve extensive washing steps. Flow cytometry based immunoassays have been known for some time and have the advantage of not needing the wash steps. Presence of a fluorescent tag on one of the components allows for detection of the resulting protein complex in a flow cytometer. However, unbound fluorescent analytes are present in the current flow cytometry based immunoassays that interfere with making quantitative measurements.

With the completion of the human genome project, the next major frontier is in the area of proteomics research. The existing assays are not sensitive enough to detect miniscule amounts of proteins. A typical proteomic analysis experiment contains the protein of interest in the femtomole to attomole range. However, existing bead based flow cytometry binding methods do not function well when the amount of protein to be detected in the sample is less than nanomole to picomole quantity.

Microfluidic devices are also important for biomolecular analysis methods. Microfluidic devices generally consume sub-microliter quantities of sample making them well suited for use when the required reagents are scarce or expensive. Because of their size, however, microfluidic devices have important practical problems delivering and mixing fluid samples.

### SUMMARY OF THE INVENTION

It is therefore an object of the present invention to provide a method sensitive enough to allow detection of femtomole to attomole amounts of biomolecule.

It is a further object to provide a means to make quantitative measurements using bead based binding assays involving no wash steps.

It is yet another object to provide a bead based method where unbound analytes do not interfere with the fluorescence resonance energy transfer measurements.

It is yet another object to provide a bead based assay, which utilizes the FLAGS system to detect epitope tagged fusion proteins, using fluorescence resonance energy transfer. (FLAG® is a registered trademark of Immunex Corp. and may be subsequently designated in this text using “FLAG” or with the symbol®.).

It is yet another object to provide a means for reducing problems with associated with the limitations of diffusion-based mixing in a channel.

Finally in all of the above embodiments, it is an object to provide a method that can be used to make dynamic real time measurements of biomolecule binding.

According to a first broad aspect of the present invention, there is provided a sensing device comprising: a vessel; a plurality of sensor beads located within the vessel to form interstitial spaces therethrough; and a plurality of biomolecules bound to at least at portion of the plurality of beads, each of the biomolecules having a fluorescent tag.

According to second broad aspect of the invention, there is provided a method for detecting the binding of two biomolecules comprising the following steps: providing a plurality of first biomolecules, each of the first biomolecules having a first fluorescent tag, each of the first biomolecules being bound to a respective substrate of a plurality of substrate; providing a plurality of second biomolecules, each of the second biomolecules having a second fluorescent tag; binding at least portion of the second biomolecules to at least a portion of the first biomolecules to form complexes, wherein the plurality of first biomolecules and the plurality of second biomolecules prior to the binding step have a pre-complexing total fluorescence and wherein the complexes and free second biomolecules after the binding step have a post-complexing total fluorescence; and detecting any difference between the pre-complexing total fluorescence and the post-complexing total fluorescence.

According to a third broad aspect of the invention, a sensing device comprising a suspension of a plurality of sensor beads; and a plurality of biomolecules bound to at least a portion of the plurality of beads, each of the biomolecules having a fluorescent tag is provided.

Other objects and features of the present invention will be apparent from the following detailed description of the preferred embodiment.

### BRIEF DESCRIPTION OF THE DRAWINGS

The invention will be described in conjunction with the accompanying drawings, in which:

FIG. 1 is an illustration showing the different ways of measuring fluorescence resonance energy transfer in accordance with an embodiment of the present invention;

FIG. 2 illustrates fluorescence resonance energy transfer measurements as an indicator of binding performed using an embodiment of the present invention;

FIG. 3 shows the progressive decrease in FRET measurement due to increasing amount of red-tagged IgG flowing through an affinity column packed with calibrated beads constructed in accordance with an embodiment of the present invention;

FIG. 4 shows reagentless detection of analyte based on FRET constructed in accordance with an embodiment of the present invention;

FIG. 5 is an illustration of binding and dissociation kinetics determination between biomolecules on beads constructed in accordance with an embodiment of the present invention;

FIG. 6 is a regenerable sensor scheme using FLAG peptide and interchangeable M1 fab fragments-detector protein complex constructed in accordance with an embodiment of the present invention;

FIGS. 7A and 7B are schematics of a microfluidic apparatus showing a configuration that may be used to deliver samples packed in microcolumns containing beads prepared in accordance with a method of the present invention;

FIG. 8 shows the equilibrium binding of TR-M1 to 5-FLAG on beads constructed in accordance with an embodiment of the present invention;

FIG. 9 shows the sigmoidal analysis of the binding of TR-M1 to 5-FLAG bearing beads in presence of calcium performed in accordance with an embodiment of the present invention;

FIG. 10 shows the sigmoidal analysis of the binding of TR-M1 to 5-FLAG bearing beads in calcium free buffers performed in accordance with an embodiment of the present invention;

FIG. 11 shows a schematic depiction of the capture of non-biotinylated fluorescent 5-FLAG peptide by biotinylated M2 IgG on beads constructed in accordance with an embodiment of the present invention;

FIG. 12 shows mean channel fluorescence of fluorescent FLAG peptide bound to M2 IgG on beads versus FLAG peptide constructed in accordance with an embodiment of the present invention;

FIG. 13 shows the standard response curves for known amounts of FLAG BAP as determined by immunoblot constructed in accordance with an embodiment of the present invention;

FIG. 14 shows the standard response curves for known amounts of FLAG BAP as determined by beads constructed in accordance with an embodiment of the present invention;

FIG. 15 shows intensity increase in fluorescence with passage of time as excess native biotin flows through the column of beads in a channel constructed in accordance with an embodiment of the present invention;

FIG. 16 shows binding curves of Texas-Red labeled monoclonal anti-FLAG antibodies passing through affinity micro-columns of fluorescein labeled FLAG peptide-bearing beads constructed in accordance with an embodiment of the present invention;

FIG. 17 is a sigmoidal dose-response binding curve of TR-M1 mAbs obtained after passage through the affinity micro-column constructed in accordance with an embodiment of the present invention;

FIG. 18 shows binding of TR-M1 mAbs to bead-borne FLAG peptides in flow cytometry in accordance with a method of the present invention; and

FIG. 19 is a table showing the characterization of binding affinities between beads, flourescein biotin, FLAG peptides, and antibodies in accordance with a method of the present invention.

### DETAILED DESCRIPTION OF THE PREFERRED EMBODIMENT

It is advantageous to define several terms before describing the invention. It should be appreciated that the following definitions are used throughout this application.

### Definitions

Where the definition of terms departs from the commonly used meaning of the term, applicant intends to utilize the definitions provided below, unless specifically indicated.

For the purposes of the present invention, the term “fluorescence resonance energy transfer” refers to the radiationless transmission of an energy quantum from its site of absorption to the site of its utilization in the molecule, or system of molecules, by resonance interaction between chromophores, over distances considerably greater than interatomic, without conversion to thermal energy, and without the donor and acceptor coming into kinetic collision. The donor is the dye that initially absorbs the energy, and the acceptor is the chromophore to which the energy is subsequently transferred.

For the purposes of the present invention, the term “biomolecule(s)” refers to peptide, small polypeptide, long polypeptide, protein, antigen, antibodies, tagged protein, oligonucleotides, nucleotides, polynucleotide, aptamer, DNA, RNA, carbohydrates, etc.

For the purposes of the present invention, the term “beads” refers to a particle that can be coated with a biomolecule. For example, a preferred bead has a range of sizes, from 0.1 μm to 1000 μm. Beads may be made of any material, such as glass, metallics, etc. Beads may be coated with any biomolecule. Beads may be in solution, in a sample, packed, in suspension, or any other suitable arrangement.

For the purposes of the present invention, the term “epitope” refers to a small polypeptide sequence that can be fused in various positions of a protein. Antibodies directed against these epitopes specifically recognize and bind to these sequences.

For the purposes of the present invention, the term “anti-epitope M1” refers to an antibody directed against the epitope present in the N-terminal position of a protein.

For the purposes of the present invention, the term “anti-epitope M2” refers to an antibody directed against the epitope present in the C-terminal position of a protein.

For the purposes of the present invention, the term “anti-epitope M5” refers to an antibody directed against the epitope present in the N-terminus, Met-N-terminus or C-terminus of a protein.

For the purposes of the present invention, the term “fluorescent tag” refers to a fluorescent molecule that can be conjugated to a biomolecule.

For the purposes of the present invention, the term “sensor bead” refers to coated beads to which a biomolecule is bound that responds to presence or absence of an analyte.

For the purposes of the present invention, the term “optically transparent material” refers to any material through which light may travel.

For the purposes of the present invention, the terms “microcolumn” and “microfluidic channel” refers to a column having a length of 5 mm to 2 cm, a breadth of 100 to 300 μm and a depth of 10 to 100 μm.

For the purposes of the present invention, the term “vessel” refers to a tube, canal, channel or container in which a fluid, sample, suspension or solution is contained, conveyed, circulated or conducted.

For the purposes of the present invention, the term “spacer beads” refers to beads in the microcolumn used to separate a sensor bead array from a neighboring different sensor array. Spacer beads may also refer to beads used to separate two adjacent arrays of beads in a microcolumn.

For the purposes of the present invention, the term “obstructive feature” refers to a feature in the microcolumn that prevents mixing of one type of sensor beads located in one sensing region of the microcolumn with other sensor beads located in a different perhaps adjacent sensing region of the same microcolumn. The obstructive feature may also be used to prevent flushing and to retain beads in the microcolumn.

For the purposes of the present invention, the term “foundation beads” refers to beads that are introduced and packed into the microcolumn before the sensor beads are packed into the same column.

### Description

The present invention relates generally to fluorescence based bead binding assays, such as assays utilizing fluorescence resonance energy transfer (FRET) as the mode of detection. Biomolecule binding on beads may be measured to quantify biomolecule sample characteristics. According to a method of the present invention there is provided a method for detecting the binding of two biomolecules including the following steps: (1) providing a plurality of first biomolecules, each of the first biomolecules having a first fluorescent tag, each of the first biomolecules being bound to a respective substrate of a plurality of substrates; (2) providing a plurality of second biomolecules, each of the second biomolecules having a second fluorescent tag; (3) binding at least a portion of the second biomolecules to at least a portion of the first biomolecules to form complexes, wherein the plurality of first biomolecules and the plurality of second biomolecules prior to the binding step have a pre-complexing total fluorescence and wherein the complexes and free second biomolecules after the binding step have a post-complexing total fluorescence; and (4) detecting any difference between the pre-complexing total fluorescence and the post-complexing total fluorescence.

Biomolecule(s) of the present invention include peptide, small polypeptide, long polypeptide, protein, antigen, antibodies, tagged protein, oligonucleotides, nucleotides, polynucleotide, aptamer, DNA, RNA, carbohydrates, etc.

Epitope tagging is an exemplary technique for studying particular types of biomolecules. Epitope tagging is a widely practiced technique used to study structure and function of new proteins. For example, purified proteins can be conjugated to small, non-protein molecules known as haptens. A protein thus tagged, can be recognized by readily available, high-affinity antibodies to the hapten. In like manner, cloned DNA, which includes a DNA sequence that encodes a known epitope, allows the resulting fusion protein to be similarly identified. Epitope tagging is particularly useful for studying new proteins for which no suitable antibodies exist.

An eight amino acid sequence biomolecule having the sequence DYKDDDDK (SEQ ID NO: 1), as shown in U.S. Pat. No. 4,851,341, may be used as an epitope and fused to ends of proteins. The fusion proteins may then be detected using three monoclonal anti-FLAG® specific antibodies provided as part of the FLAG® system. (FLAG® and anti-FLAG® are registered trademarks of Immunex Corp. and may be subsequently designated in this text using “FLAG” or “anti-FLAG” or with the symbol®). A fusion protein containing a FLAG epitope is readily amenable to studies involving protein-protein interactions. The entire disclosure and contents of U.S. Pat. No. 4,851,341 is hereby incorporated by reference.

The present invention is representative of the development of a quantitative bead based high throughput biomolecule tagged binding assay. For example, the assay may utilize an epitope containing the amino acid sequence DYKDDDDK (SEQ ID NO: 1) “flag” described in U.S. Pat. No. 4,851,341. This FLAG epitope is widely used for purification and detection of fusion proteins. The role of the FLAG peptide as a universal marker of fusion proteins is facilitated by the fact that it is made up of both hydrophilic and hydrophobic residues. This combination ostensibly enables the FLAG sequence to remain generally accessible to antibodies even when bound to relatively large proteins. In typical applications, FLAG may be used to purify proteins and to study protein interactions, protein structure, or protein localization. For purification and detection of fusion proteins, the FLAG system uses three monoclonal anti-FLAG antibodies. Each antibody recognizes and binds to the FLAG epitope with different specificities that depend on the position of the FLAG peptide in the fusion protein: Anti-FLAG M1 specifically binds to fusion proteins with the FLAG epitope at the free N-terminus. Binding of the M1 antibody is calcium dependent. Anti-FLAG M2 is calcium independent and reacts with fusion proteins with the FLAG epitope at the N-terminus, Met-N-terminus (MDYKDDDDK (SEQ ID NO: 2)) or C-terminus. Anti-FLAG M5 recognizes the N-terminal Met-FLAG fusion proteins, and its binding is not dependent on calcium. Thus, epitope tagged proteins can then be effectively subjected to techniques such as affinity chromatography, immuno-blotting, immuno-precipitation, and immuno-fluorescence. These immunoassays are normally time consuming.

Standard microplate based sandwich immunoassays such as ELISAs are time consuming and involve extensive washing steps. Flow cytometry based immunoassays have been known for some time and have the advantage of not needing wash steps. Presence of a fluorescent tag on one of the components allows for detection of the resulting protein complex in a flow cytometer.

The existing sandwich based assays utilizing flow cytometry to detect bead binding, as described in U.S. Pat. No. 6,159,748, are fraught with several disadvantages. The entire disclosure and contents of U.S. Pat. No. 6,159,748 is hereby incorporated by reference. The existing assays require multiple wash steps and only enable semiquantitative measurements. Furthermore, the results obtained from the existing bead based assays are indirect. The existing systems have an antigen bound to the bead. A primary antibody directed against the antigen makes a complex with the bead. A secondary antibody conjugated with a fluorescent tag recognizes and binds to the primary antibody in the bead-antibody complex. The resulting fluorescent complex is detected using a flow cytometer. With the existing bead based methods, one is limited to making only end point binding measurements. The end point assay containing the fluorescent tag on the secondary body has the additional disadvantage that it is not very sensitive because fluorophore tags on secondary antibodies tend to have low emission yields as compared to native fluorophores. Hence, a sensitive method that could provide real time measurements of biomolecule interactions would help the efforts in the emerging genomic and proteomic research fields.

The present invention utilizes the existing epitope tag methodologies and makes them available for use generally with biomolecules in the area of flow cytometry research. The current invention extends these techniques into the area of fluorescence and adapts the epitope system for use as a key component in bead based analytical and fundamental studies involving biomolecule interactions.

The present invention requires no wash steps and provides quantitative measurements of dynamic real time binding interactions occurring between biomolecules, such as proteins. Another embodiment of the invention may be reagentless. Yet another embodiment of the present invention may be regenerable. Ultimately, the methodology of the present invention is described as a general assay applicable to other proteomic assays.

The present invention also describes bead-based assays, which utilize the FLAG system to detect epitope tagged fusion proteins, by fluorescence methods such as fluorescence resonance energy transfer (FRET), using flow cytometry.

FRET is a distance-dependent interaction between the electronic excited states of two dye molecules in which excitation is transferred from an excited donor molecule to an acceptor molecule without emission of a photon. The absorption spectrum of the acceptor must overlap the fluorescence emission spectrum of the donor. FRET, between donor and acceptor, occurs over distances that typically span distances in the range 10-100 Å. The characteristic distance at which the donor fluorescence and FRET are equally probable is defined as R₀:

R₀=9.79×10³(κ²n⁻⁴φ_{D}J)⁻⁶Å

Where n is the refractive index of the medium, J is the spectral overlap between donor and acceptor, φ_{D} is the emission quantum yield of the donor, and κ² is the orientation factor between donor and acceptor.

As described above, the distance separating the donor and the acceptor plays an important role in FRET. If the acceptor molecule is not close enough to the donor molecule then the energy that is emitted from the donor cannot be absorbed by the acceptor and is emitted as a photon and no FRET occurs. For FRET to occur clearly, the following requirements have to be met:

1. The donor probe must have a high emission quantum yield.

2. The emission spectrum of the donor probe must overlap considerably the absorption spectrum of the acceptor probe.

3. There is an appropriate alignment of the absorption and emission moments and their separation vector.

4. The donor and acceptor must be within 0.1±1.9×R₀ from each other. If fluorescein is used as a donor, the distance in which FRET occurs varies according to the acceptor molecule. In the examples described later in the application, D/A pair is comprised of fluorescein tagged FLAG peptides and Texas-Red labeled monoclonal antibodies. An R₀ value for this D/A pair was determined to be on the order of 45 Å from a numerical solution of the spectral overlap integral (I) using normalized donor emission and acceptor spectral data. The probability of FRET is optimized herein by use of antibodies with relatively high densities (≈6.0) of energy acceptors (A).

5. The acceptor probe may be fluorescent or non-fluorescent.

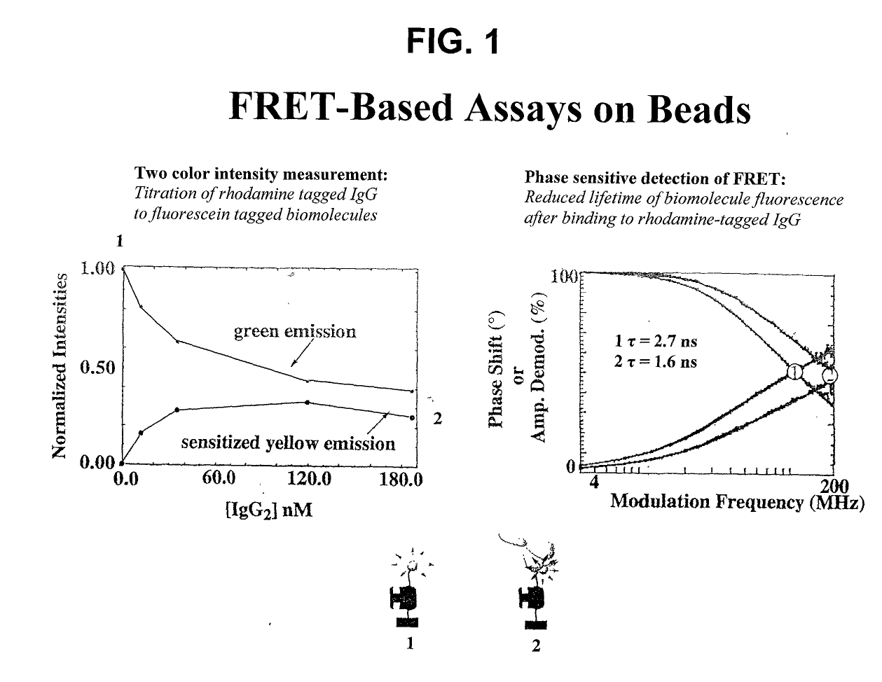

FIG. 1 is a schematic illustrating two ways of measuring FRET-based bead binding assays. FRET measurements can be made by two color intensity measurements or by detecting changes in the lifetime of the donor.

Two Color Intensity Measurements

A green fluorescein labeled biomolecule is tethered to the beads. The total fluorescence measured in presence of only a green labeled biomolecule is normalized to 1. Now rhodamine tagged antibodies represented by IgG that recognize and bind to the fluorescein labeled biomolecule are added. As the concentration of rhodamine labeled IgG increases in the system, the binding of antibody to the biomolecule brings the fluorescein label close to the rhodamine label of the antibody, causing the FRET and resulting sensitized yellow emission. At 0 nM IgG₂ there is no yellow emission. As the concentration of rhodamine labeled IgG increases, the green emission decreases and yellow emission increases due to binding of antibody to the biomolecule resulting in FRET between green and red fluorescent tags resulting in yellow emission.

The binding of rhodamine tagged IgG to the fluorescein labeled biomolecule reduces the lifetime of the biomolecule fluorescence.

Flow cytometry is a sensitive and quantitative method for measuring fluorescence or light scatter of particles. The detection of binding interactions associated with the particle surfaces forms the basis of measurements relevant to many assays. These include steady state and kinetic analysis of ligand binding and enzyme activity. Flow cytometry based immunoassays are similar in concept to micro-plate based ELISA or sandwich assays, with the advantage that wash steps are often not needed.

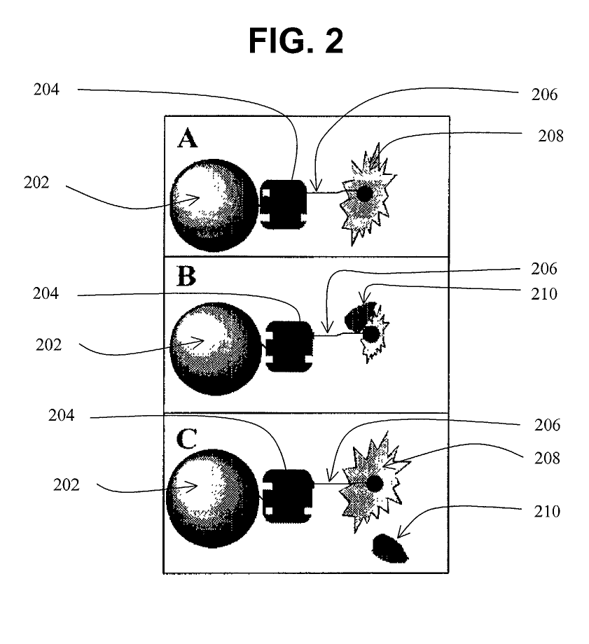

FIG. 2 is a schematic illustrating the use of FRET as an indicator of binding. The schematic in FIG. 2 may be formed using any suitable biomolecule. For example, Panel A may show bead 202 coated with streptavidin 204. FLAG peptide 206 carries a fluorescein tag 208 on one end and is tethered to bead 202 through streptavidin 204 at the other end. The streptavidin coated bead is calibrated so that the surface coverage of peptide 206 on its surface is known.

Texas-Red labeled antibodies 210 are antibodies raised against the FLAG epitope of peptide 206. As shown in FIG. 2 panel B, antibody 210 binds to peptide 206. Binding of antibody 210 to peptide 206 brings the Texas-Red fluorescent label 210 in close proximity of green fluorescent tag 208 to cause FRET. Since the initial fluorescence with the fluorescein tagged FLAG peptide 206 bound to the streptavidin coated bead was known, binding of Texas-Red antibody 210 to peptide 206 gives a FRET signal of known calibration.

The presence of analyte (an unlabeled antibody on an epitope tag) (not shown), blocks the access of antibody 210 to the FLAG epitope of peptide 206. Hence, in presence of this analyte, some of the epitopes will not be available for binding of antibody 210. As the amount of analyte is increased, more of the epitopes will be unavailable for binding of antibody 210. As shown in FIG. 2 panel C, less of antibody 210 will be present close to fluorescein tag 208, resulting in an altered FRET signal. The FRET signal will be altered in proportion to the amount of analyte present. As more analyte is added to the system, less FRET will be measured and vice versa.

The use of FRET as an indicator of binding has some advantages over fluorescence measurements. First, because the fluorescence of the beads used in FRET corresponds to a known concentration of surface receptors, therefore, the subsequent changes define the amount of captured analytes, without signal interference from unbound analytes. Second, FRET based assays are amenable to the development of reagentless assays where both biomolecule and antibody are tethered to the surface. The overall sensitivity of these assays is related to the number of assemblies per bead and the number of beads for precise detection for effective application of quantitative flow cytometry. Thus, the immediate advantage presented over existing methods includes increased sensitivity.

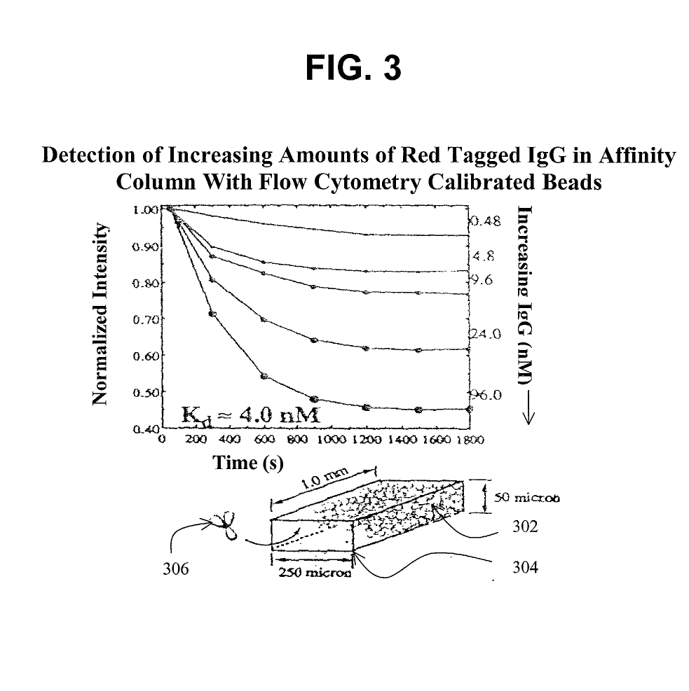

Another embodiment of the present invention shown in FIG. 3, may use calibrated beads suitable for use in flow cytometry. These beads coated with antigen 302 containing a fluorescein tag are packed into an affinity column 304 having a width of 250 μm, length of 1.0 mm and a height of 50 μm. Initial fluorescence of the calibrated beads in the column is normalized to 1.0. As increasing amounts of red-tagged IgG 306 having affinity for the green-tagged antigen is added to the affinity column, the binding of antibody 306 to antigen 302 brings the red tag in close proximity of the green tag resulting in FRET. As shown in FIG. 3, the initial fluorescence was normalized to 1.0. Binding of red-tagged IgG 306 reduces the net green fluorescence emitting from the column and the normalized intensity of fluorescence falls below 1.0. This embodiment of the present invention is very sensitive and as shown in FIG. 3 can detect miniscule amounts of protein down to the femtomole-attomole range.

The present invention tethers the fluorescently tagged biomolecule to the bead. Thereby the biomolecule of unknown function fused to, for example the FLAG epitope, is anchored to the beads. The present invention provides a convenient system whereby the fusion tagged biomolecules can be detected in unprecedented trace quantities from crude extracts or culture supernatants.

Another embodiment of the present invention may use an antigen/antibody system that is completely different from the FLAG system. Yet another embodiment may use a different pair of fluorescent donor and acceptor molecules other than green fluorescein and red rhodamine. Other fluorescent green tags that may be used in the present system are BODIPY™ and ALEXA™ series of dyes from Molecular Probes.

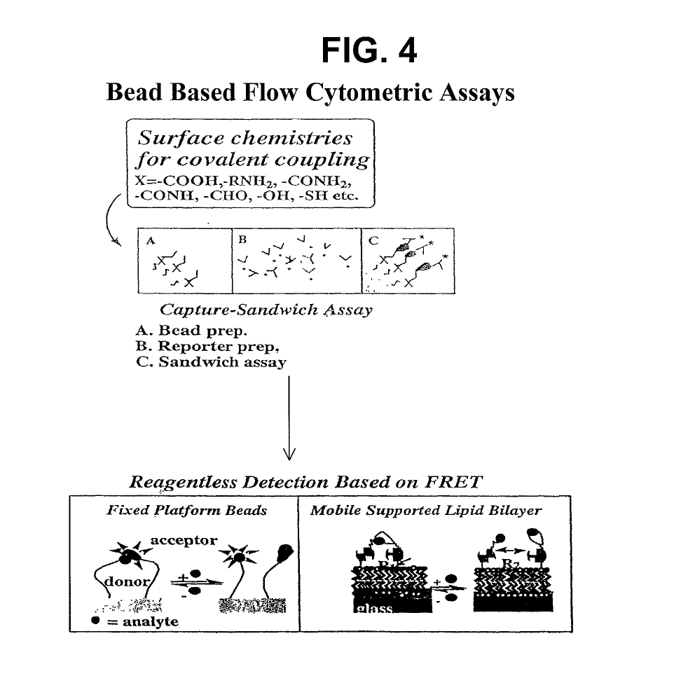

FIG. 4 is a schematic illustrating how different surface chemistries may be used for coupling the biomolecules to the beads. The biomolecules may be coupled using —COOH, —RNH₂, —CONH₂, —CONH, —CHO, —OH, —SH groups, etc. Beads that have biomolecules coupled to them are used to develop a system that provides a means to perform reagentless detection of presence of an analyte based on FRET.

In one embodiment of the reagentless detection of analyte based on FRET, the biomolecule labeled with a donor fluorescent tag and an antibody labeled with an acceptor fluorescent tag are fixed to the same platform as shown in FIG. 4. The antibody directed against the epitope on the labeled biomolecule recognizes and binds to the biomolecule. The donor and acceptor fluorescent tags are brought in close proximity resulting in FRET. A crude extract containing analyte is added to the system. If the analyte blocks the access of the antibody to the antigen or the analyte covers the recognition epitope of the antigen, the antibody is unable to bind to the antigen. Under these conditions, a certain fraction of the acceptor molecules will not be bound to the donor molecules resulting in decreased FRET. The decrease in FRET will be proportional to the concentration of analyte present in the crude extract that is able to disrupt the antigen/antibody binding. Thus, presence of trace levels of analyte results in decreased FRET measurements.

The above-described reagentless detection of analyte based on a FRET system may be used as a sensor system for quality control purposes. Here the aim is to ensure that each batch of solution is free of a certain solute. If this solution is introduced into an appropriately engineered system where the solute to be detected serves the same purpose as the analyte described above, a decrease in FRET would indicate presence of the solute. The amount may be quantified, and based on that data, the quality control manager may decide the appropriate course of action.

For some other biological purposes, the present invention envisages yet another embodiment of the present invention where the beads are mobile and supported on a lipid bilayer instead of a fixed support as shown in FIG. 4.

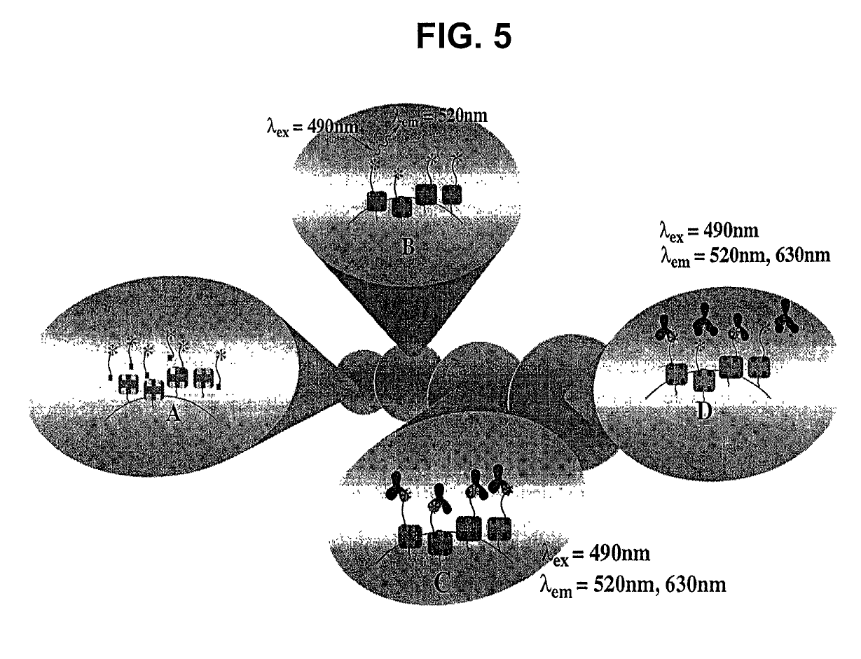

Another embodiment of this invention facilitates the study of the dynamic interactions between novel proteins thus enabling the resolution of their binding and dissociation kinetics. The binding and dissociation kinetics between proteins on beads is illustrated in FIG. 5. The system enables the use of biomolecules, such as FLAG tagged proteins, in mediating the determination of binding affinities of new proteins. A protein labeled is sequestered to beads via biotin or His-tag tether. Subsequently, the binding and dissociation of a second protein labeled is analyzed with the aid of a fluorescent antibody fragment bound to the flag epitope tag. Thus, in this invention the binding of a fluorescent FLAG antibody to the bead is a measure of the binding event and removal of the fluorescent FLAG antibody from the bead is a measure of the dissociation.

The present invention also employs other characteristics of the general theme of sequestering proteins to beads as part of a sensing scheme. Proteins or small molecule ligands are tethered using biotin to streptavidin-bearing beads made of polysterene or lipid, or His-tags to Ni-coated or Nickel chelating lipobeads.

Another embodiment of the present invention may be used to develop a regenerable assay using ion sensitive ligands. In this system, a suitably epitope tagged peptide may be tethered to the beads. A system such as a FLAG tagged peptide that binds to the receptors only in presence of Ca²⁺ is tethered to the beads. The assay is performed in the presence of Ca²⁺. Analyte is introduced into the system in presence of Ca²⁺ and the analyte binds to the FLAG peptide. FRET is measured to determine the amount of analyte bound. Once the assay is completed, Ethylenediaminetetraacetic acid (EDTA) is used to remove the Ca²⁺ from the system. EDTA is a chelating agent that sequesters and removes Ca²⁺. In absence of Ca²⁺ the FLAG peptide releases the analyte, thereby renewing the sensor for a second round of analysis.

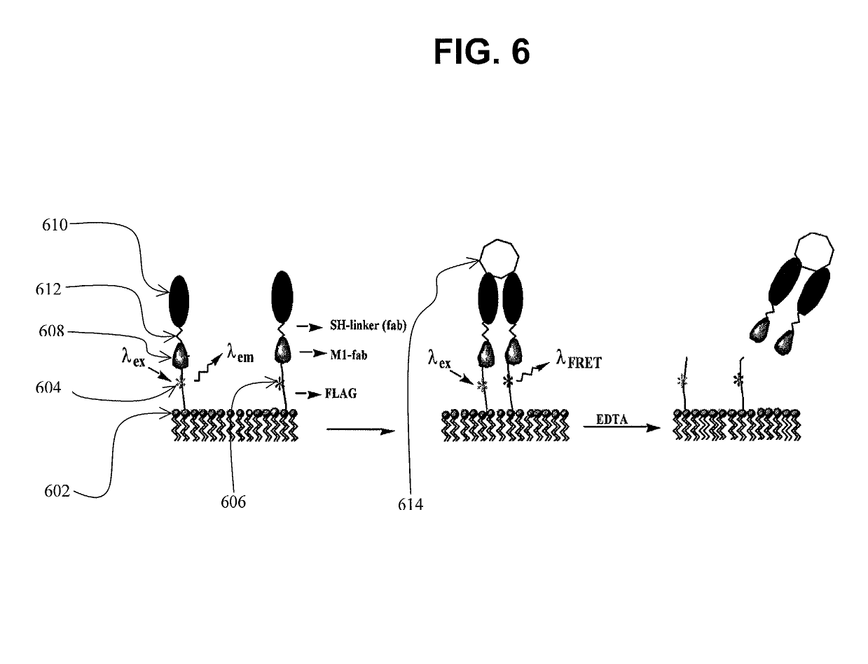

Another embodiment of the present invention comprising a molecular assembly leading to regenerable sensor surfaces based on FRET is shown in FIG. 6. Here the transducer surface of a sensor that comprises beads 602. FLAG peptide tagged with green fluorophore 604 and FLAG peptide tagged with red fluorophore 606 may be tethered to beads 602. Binding of M1 fab fragment 608 is calcium dependent. In presence of calcium, FLAG peptides 604 and 606 have fragment 608 bound to them. Protein 610 is fused to fragment 608 via a fab SH-linker 612. Analyte 614 binds to protein 610 in a multi-step process. Stable binding of analyte 614 occurs when analyte 614 serves as a bridge between a pair of protein 610 molecules. The stable binding of analyte 614 brings the FLAG peptides 604 and 606 into close proximity leading to FRET. Thus, the FRET signal is indicative of stable binding of analyte.

Transducer sensor surface of the present invention is regenerated by introduction of EDTA. EDTA chelates divalent cations and depletes calcium from the system. In absence of calcium, fragment 608 dissociates from peptides 604 and 606. Analyte 614 is stably bound to a pair of proteins 610 linked to fragment 608 via linker 612. Therefore, dissociation of fragment 608 results in removal of the entire complex. Thus, depletion of calcium from the system allows for the facile regeneration of the transducer platform. The same transducer platform can be used over and over again for multiple assays using the same protein pair-analyte combination or it can be used for different assays using different protein-pair-analyte combinations.

Thus, the advantage of the present invention over existing methods includes, time, increased sensitivity, kinetic resolution of the binding process, as well as ease of use. The approach is compatible with high throughput flow cytometry, a method in which submicroliter samples from multiwell plates are analyzed at rates up to 100 samples per minute.

Microcolumn Sensors

Important progress in the development of new technologies for biomolecular analysis has been made, in particular, in the area of microfluidic devices. Microfluidic devices generally consume sub-microliter quantities of sample and are thus well suited for use when the required reagents are scarce or expensive. Because of their size, microfluidic devices operate in a regime where small Reynolds numbers govern the delivery of fluid samples. Fast mixing of reagents is one of several issues that present a major challenge to the operation of microfluidic devices. Due to negligible inertial forces, mixing of solutes in microchannels is as a rule driven by diffusion alone, and is therefore slow and often ineffective even at micrometer scales. Other factors including fluid transport and quantitative analysis such as chemical reaction, product separation and identification etc. of molecular interactions are poorly understood and must be optimized to fully realize the potential of these micro-devices.

A prevailing trend in the development of bioanalytical assays, is the display of biochemical reagents on synthetic microbeads. Important progress has been made in the incorporation of microbeads in sensor arrays whose functions are based on the natural sensory functions of smell and taste. Efforts directed towards developing biosensing strategies that display fluorescently labeled receptors and ligands on microbeads resulted in development of the know how to produce molecular assemblies on beads that may be analyzed in a quantitative fashion by flow cytometry.

In the present invention, beads calibrated with flow cytometry serve as platforms in an affinity micro-column format for the quantitative detection of analytes in microfluidic channels. There are several advantages to this approach: molecular assemblies for the assay are created outside the channel on beads and calibrated with flow cytometry; uniform populations of beads may be insured through rapid cytometric sorting; and beads present a larger surface area for the display of receptors than flat surfaces. These are clear improvements over those techniques that rely on the micro patterning of reactive molecules on flat surfaces. Mixing of solutes in laminar flows occurs by diffusion with a typical diffusion coefficient for biomolecules on the order of ≦10⁻⁷ cm² s⁻¹. Thus, mixing by diffusion is slow. Rapid mixing in the microcolumn is achieved because the distance that must be covered by diffusion is limited to the small interstitial space between the closely packed receptor-bearing beads. Analytes are captured in flow-through format and as such each bead can act as a local concentrator of analytes. Beads can be easily configured to detect multiple analytes in the restricted confines of a microchannel. Simultaneous detection of a diverse group of analytes can be achieved by packing discrete segments of receptor bearing beads in a single affinity microcolumn-system.

Samples of Texas-Red labeled anti-FLAG monoclonal antibodies referred to as TR-M1 mAbs were pumped through an affinity microcolumn with fluorescein-tagged FLAG peptides on beads with known site densities. The interaction between the TR-M1 mAbs and beads was monitored via FRET. Monitoring the amount of ligand/receptor complex formed at a wide range of concentrations of TR-M1 mAbs gave access to the kinetic and equilibrium parameters of the antibody-biomolecule reaction. The data from affinity micro-columns were compared to data measured in a conventional flow cytometer assay.

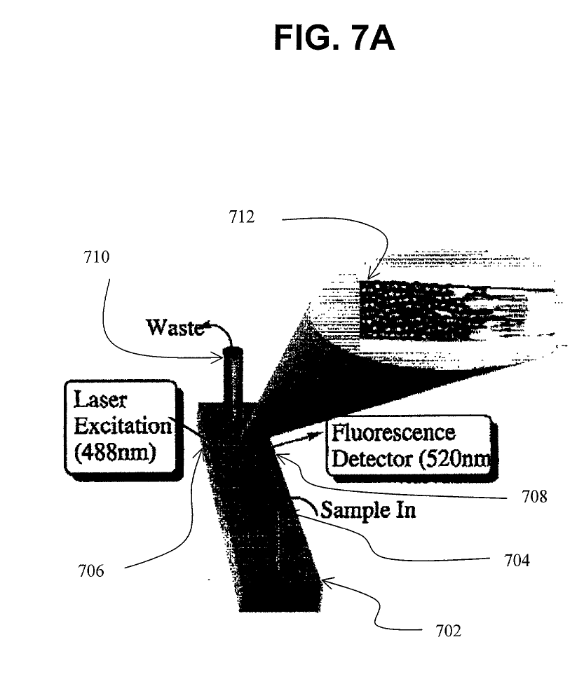

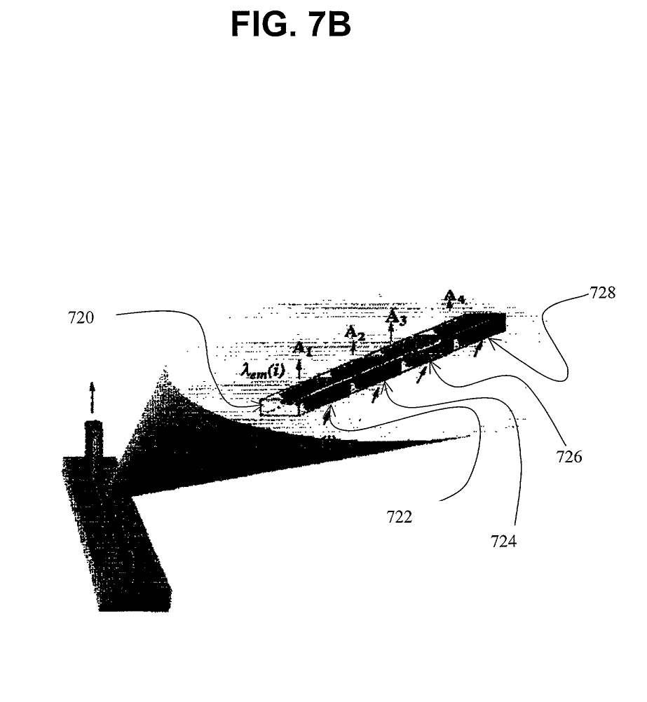

A schematic of a microfluidic apparatus showing a configuration that may be used to deliver samples packed in microcolumns containing beads prepared in accordance with a method of the present invention is shown in FIG. 7A. The device comprises a box 702. Elastomeric silicone microchannel (not shown), is mounted on a glass slide with two openings for sample delivery and egress. The micro-channel may be, for example, approximately 250 μm wide, approximately 50 μm deep and approximately 3 cm long. The samples may be introduced from entry port 704. The samples contained in the microchannel may be exposed to a laser beam of 488 nm. Excitation laser source 706 is located on one side of the microchannel. A fluorescence detector 708 is located to the other side of the microchannel facing the excitation laser source. The orientation of the microchannel is such that all the beads packed in the microcolumn are exposed to the excitation laser. The resulting FRET is detected using detector 708. At the distal end of box 702 is an outlet 710 through which waste exits the microfluidic device 710. Patterned features shown in inset 712 are spaced 20 μm apart. These act as filters for holding the beads in place. In a preferred embodiment, thirty thousand 6.2 μm streptavidin coated beads form a 600 μm long affinity microcolumn. The 6.2 μm beads may be made of polystyrene, glass, etc. The sample is delivered and fluorescence measurements taken with a spectrofluorimeter.

In a preferred embodiment of the present invention, the microfluidic channels are made from an elastomeric polymer, such as poly(dimethylsiloxane) (PDMS), where convenient fabrication techniques allow for dimensions as small as 10 μm.

The prototype shown in FIG. 7A may be composed of a microfluidic channel with dimensions of approximately 3 cm long, with typical dimensions of approximately 250 μm by approximately 50 μm in breadth and depth, patterned into a PDMS elastomer adhered to a glass slide support. Surface calibrated beads were sequestered in the channels and used as platforms for the dynamic and quantitative detection of biomolecules at sub-microliter volumes. Within the microchannel, obstructive features, 20 μm apart were patterned as filters to hold 30 μm beads. Beads were packed by injection of suspensions, starting with a foundation of 30 μm borosilicate beads followed by the affinity micro-column layer of thirty thousand, 6.2 μm streptavidin-coated beads. The streptavidin coated beads bore biotinylated molecules of interest. The void space or the interstitial bead space within the bioactive 600 μm column is reduced to ≈4.0 nL and serves as the reactor vessel with an intrinsically large surface area.

Another embodiment of the prototype microfluidic device is shown in FIG. 7B. In this embodiment, a model multi-analyte detection array microcolumn 720 is depicted. Column 720 can be visualized as being segmented into several affinity micro-column arrays 722, 724, 726 and 728. Each array 722, 724, 726, 728 comprises beads bearing receptors for different analytes A₁, A₂, A₃ and A₄ respectively. Each array in microcolumn 720 may be associated with differently tagged receptors to be interrogated at given excitation wavelengths represented by λₑₓ and corresponding given emission wavelength λₑₘ. Array 722 may have an λₑₓ₁ and λₑₘ₁, array 724 may have an λₑₓ₂ and λₑₘ₂, array 726 may have an λₑₓ₃ and λₑₘ₃ and array 728 may have an λₑₓ₄ and λₑₘ₄ respectively associated with them.

A multi-analyte model system comprised of discrete segments of beads that bear distinct receptors for the simultaneous detection of diverse analytes has been developed. Proof of concept data has so far been obtained from an affinity column bearing two segments of distinct receptor bearing beads. Since these assays consume very small sample volumes, multiple tests can be run, therefore saving on expensive reagents.

Another embodiment of such a microcolumn may be used to assay multiple analytes simultaneously. Yet another embodiment of the present invention may include parallel microfluidic networks, with individual sample delivery ports or a single one with several downstream branches.

Affinity Immunoassays

The approach to biomolecular assemblies displayed on microbead based affinity columns has features in common with competitive binding immunoassays and affinity chromatography. In these formats, a fluorescently tagged analyte analogue is incubated with a fixed amount of a dark target analyte and applied to a column that bears antibodies that can bind to both of these species. This is usually done by simultaneously or sequentially injecting the target analyte and its labeled analogue onto the column. The result is a method known as a chromatographic or flow injection immunoassay. The generation of a signal is due to the presence of a target analyte in the sample that causes a change in the amount of labeled analyte that is able to bind to the antibodies in the system. A signal that corresponds to the target analyte's concentration is acquired by either measuring the amount of the labeled analyte that elutes in the non-retained peak or analysis of the bound labeled analyte that is released when an appropriate elution buffer is applied to the column.

In the present invention, the beads bear fluorescent ligands/receptors of known surface occupancy. Thus, the subsequent changes from the initial intensity reading bear definite and known relationships to the amount of captured analytes, without contribution from unbound species. Thus direct analysis of the fate of the analyte species during passage through the affinity micro-column may be performed in real time.

The present invention will now be described with help of examples.

### EXAMPLES

Characterization of Molecular Assembly Components

Biotinylated and Fluorescein Tagged FLAG Peptides and Fluorescently Tagged Antibodies

The fluorescent labeling of the FLAG peptides may be achieved by using a fluorescein isothiocyanate lysine conjugate derived from a mixture of fluorescein isomers at the 5- and 6-positions of fluorescein's “lower” ring also commonly known as isomers I and II respectively. Though the spectra of the two isomers are almost indistinguishable in both wavelength and intensity, the isomers may differ in the geometry of their binding to proteins, and the conjugates may elute under different chromatographic conditions. Thus, under reverse-phase HPLC purification, two peaks putatively corresponding to fluorescein's isomers 5- and 6-position labeled FLAG peptides at peaks eluting at 12.861 and 13.312 minutes may be resolved and collected. The 5- and 6-isomers of carboxyfluorescein have been temporally resolved via HPLC analysis with the 6-preceding the 5-isomer. The temporal order of elution was supplied by Anaspec (San Jose, Calif.)—the commercial source of the Fmoc-Lys(fluorescein)-OH. This is consistent with the result that has been reported for the separation by HPLC of 5- and 6-isomers of carboxyfluorescein. Further details of the molecular assembly component characterization can be found in the following reference: Tione Buranda, Gabriel Lopez, Peter Simons, Andrzej Pastuszyn and Larry Sklar, “Detection of Epitope-Tagged Proteins in Flow Cytometry: FRET Based Assays on Beads with Femto-mole Resolution” Analytical Biochemistry, Volume 298, No. 2, 2001 (in press), the entire contents and disclosure of which is hereby incorporated by reference.

Spectroscopic Characterization of the Synthesized 6-FLAG and 5-FLAG Peptides

Spectrofluorimetric measurements were performed in single photon counting mode on an SLM-Aminco 8000 spectrofluorimeter obtained from SLM Instruments, Rochester, N.Y. The sample was excited at 490 nm, with a 10 nm band pass interference filter made by Corion Corp., Holliston, Mass. was used for line narrowing and stray light rejection. Fluorescein emission was monitored at 520 nm via a long-pass band filter 3-70 Kopp obtained from Glass, Pittsburgh, Pa. and a 520 nm also referred to as 10 nm bandpass filter obtained from Corion Corp. Neutral density filters were used to keep light intensities of the brightest samples within the dynamic range of the phototube.

Labeling of Anti-FLAG Monoclonal IgG

For fluorescein labeling, 5 mg M1 IgG in 0.5 ml sodium bicarbonate buffer at pH 8.3 was reacted with 50 μL of 1 mg/ml fluorescein-NHS obtained from Pierce, Rockford, Ill., in DMSO for two hours at room temperature. The antibody was freed of unreacted fluorescein-NHS by size exclusion chromatography using Sephadex G-25, 20-80 μm; supplied by Sigma and concentrated by ultra filtration from phosphate buffered saline using a 10,000 NMWCO Centricon membrane.

For Texas-Red labeling, 5 mg M1 IgG in 0.5 ml sodium bicarbonate buffer was reacted with 50 μL of 1 mg/ml Texas-NHS obtained from Molecular Probes, Eugene, Oreg., in DMSO for 2 hours in the dark at room temperature. The antibody was freed of unreacted Texas-NHS by dialyzing the sample using mini dialysis tubes supplied by Pierce. It is noted that because Texas-Red labeled proteins tend to stick to chromatographic columns, the sample was purified and concentrated by ultra filtration from phosphate buffered saline using a 10,000 NMWCO Centricon membrane. The fluorophore to protein (f/p) ratios were determined following standard procedures from the manufacturers. The f/p ratios were generally on the order of 6:1.

The chemical labeling of the M1 antibody with Texas-Red fluorophores resulted in an average of ≈6.0 fluorophores/antibody with negligible loss in antibody activity. The high density of Texas-Red fluorophores per antibody favors the likelihood of having at least one Texas-Red moiety in close proximity to the fluorescein tag on the FLAG peptide. Thus, enhancing the effectiveness of FRET as a transducer of the binding of antibodies to biomolecules.

Determination of the Binding Affinities of the Biotinylated FLAG Peptides and Antibodies to Streptavidin-Coated Beads

Binding analysis of biotinylated biomolecules to streptavidin-bearing beads were performed. Centrifugation assays using paired spectrofluorimetric and flow cytometric analysis were carried out to compare and corroborate the flow cytometric data against the traditional spectroflurometric measurements.

The flow cytometric analysis used a Becton-Dickinson FACScan flow cytometer obtained from Sunnyvale, Calif. that interfaced to a Power PC Macintosh using the CellQuest software package. The FACScan is equipped with a 15 mW air-cooled argon ion laser. The laser output is fixed at 488 nm. It has been shown that the mean of the histogram is the quantity relevant to binding capacity. The average fluorescence of a single bead is converted to the number of fluorophores per bead on the basis of flow cytometric calibration beads obtained from Quantum 825 Flow Cytometry Standards Corporation, San Juan, PR. Conversion from mean channel fluorescence of histograms to the total concentration of bound ligand for e.g. using fluorescein biotin or FLAG peptide, [L]_{b} is shown in equation below.

$${[ L ]}_{b} = \frac{{MCF}_{{L}_{b}}}{{MCF}_{std}} \cdot MESF \cdot \frac{1}{{Φ}_{L}} \cdot \frac{1}{{φ}_{b}} \cdot \frac{n}{A}$$

MCF_{Lb} mean channel fluorescence are the means of the histograms of the bound ligand L_{b}, corrected for nonspecific binding and standard calibration beads referred to as std, observed at similar detection settings. MESF stands for mean equivalent of soluble fluorophores and is the number of fluorescein molecules whose emission intensity is equal to MCF_{std} on each bead. The MESF is based on native fluorescein. Φ_{L} is the emission yield of unbound fluorescein biotin relative to native fluorescein. φ_{b} is the quantum yield of bound relative to free fluorophores, and is dependent on ligand type as well as the extent of surface coverage; n is the number of beads per liter and A is Avogadro's number.

Methods used for Standardization of the Beads Used in the Present Invention

Compilation of a set of K_{d} Values

Different experiments that were done to determine the Kd values are listed below:

1. Kinetic Cytometric Analysis.

2. Binding of Biotinylated M2 IgG to Beads.

3. Binding Affinity of FLAG Peptide 2 to Bead-Borne M2 Antibodies.

4. FRET Assay of M1 and FLAG Peptide 2 Binding in Solution.

5. M1 and Peptide 3 FLAG Affinity on Beads.

6. Determination of K_{d} from FRET Data.

Details about how these experiments were conducted are enumerated below.

Kinetic Cytometric Analysis

The time course of association was measured for the binding of fluorescein-labeled M1 IgG and beads bearing non-fluorescent FLAG peptide 1. In a typical run, a 15-second baseline was collected before an aliquot of fluorescent M1 was added to the sample, and resuming data collection to completion at some desired time for, e.g. 500 seconds. Dissociation rates were measured by interrupting a binding experiment after a given time, e.g. 500 seconds, then adding excess EDTA, and resuming data collection.

Binding of Biotinylated M2 IgG to Beads

Non-fluorescent biotinylated M2 antibodies were incubated with ≈1×10⁶ beads/ml in 400 μL volumes in concentrations ranging from 0.3 nM-100 nM. The bead suspensions were then centrifuged and resuspended in buffer three times to remove excess antibody. Subsequently, the M2-bearing bead samples were split into pairs, with one of the pair mixed with non-biotinylated fluorescent FLAG peptide 206 shown in FIG. 2 and the other having a thousand-fold excess of non-fluorescent biomolecule. After an hour, the paired samples were centrifuged and the fluorescence intensity of the residual supernatants measured, thus determining the binding capacity of the biotinylated M2 antibody.

The biotinylated M2 IgG from Sigma has a reported average of 7 biotin groups linked to the Fc portion of each antibody. The multivalency can be inferred to lead to very tightly bound IgGs as well as fewer M2/bead at maximum occupancy compared to the monovalent FLAG peptides. From centrifugation data, the M2 bearing beads are estimated to have a maximum site-occupancy of >1 million antibodies/bead, given the bivalent nature of antibodies, the number of receptors is doubled.

Binding Affinity of FLAG Peptide 2 to Bead-borne M2 Antibodies

Concentrations in the range of 1.38 nM-54.8 nM were used. Non-biotinylated fluorescent FLAG peptide in different concentrations were incubated in 400 μL volume suspensions of M2 bearing beads. The resulting mixture had ≈1×10⁶ beads/ml and 3.88×10⁵ M2/bead. After an hour the samples were centrifuged, and the residual supernatants and the beads were analyzed on the spectrofluorimeter and the flow cytometer respectively.

FRET Assay of M1 and FLAG Peptide 2 Binding in Solution

A 400 μL aliquot of 1.0 nM FLAG peptide in Tris buffer with 1 mM Ca²⁺ was placed in a cylindrical cuvette supplied by SIENCO Inc., Morrison, Colo. for measurements in single photon counting mode on an SLM-Aminco 8000 obtained from SLM Instruments, Rochester, N.Y. After taking the initial fluorescence spectrum measurement of FLAG peptide, 0.40 μM of Texas-Red tagged M1 IgG labeled as TR-M1 was titrated in 2 μL volumes into the stirred cuvette through the top of the sample compartment using Hamilton syringes obtained from Reno, Nev. Binding of the antibody and concomitant quenching, via FRET, of peptide fluorescence was rapid, with the peptide intensity leveling off within a minute. The change in intensity with each addition of antibody was monitored continuously until the endpoint was reached.

M1 and Peptide 3 FLAG Affinity on Beads

A stock suspension of beads bearing ≈1×10⁶ biomolecules/bead was incubated for 30 seconds with native biotin to undo ostrich quenching. The beads were then centrifuged and resuspended in buffer, repeating this process five times, to remove the excess native biotin. 25 μL volumes of bead suspensions containing 7.2×10³ beads each; thus [FLAG]≈0.5 nM were added to microfuge tubes. Texas-Red labeled M1 was then added, in 1-2 μL volumes to obtain final concentrations of 0 nM, 1.0 nM, 3.0 nM, 10.0 nM, 30.0 nM, 100.0 nM and 300.0 nM in the respective tubes. The samples were incubated for 30 minutes with shaking, followed by transfer to FACScan tubes with buffer added to the tubes to a final volume of 200 μL for flow cytometry analysis. Data was also collected in Ca²⁺ free buffer.

Determination of K_{d} from FRET Data

The titration of Texas-Red tagged IgG into a solution of FLAG peptide of initial intensity, I₀, results in a series of recorded intensities, Iᵢ, corresponding to each titration step until the final intensity is reached, If. For each titration step, the bound IgG ([L]_{b}) can be determined from the equation shown below.

$${[ L ]}_{b} = \frac{{I}_{0} - {I}_{i}}{{I}_{0} - {I}_{f}} \cdot {[ FLAG ]}_{i}$$

The K_{d} is determined from the sigmoidal plot of intensity changes,

$$\frac{{I}_{0} - {I}_{i}}{{I}_{0} - {I}_{f}} ,$$

versus the log of the concentration of the free antibody. The analysis and fits of all data were done using the software package GraphPad Prism supplied by GraphPad Software, San Diego, Calif.

5-FLAG on Beads

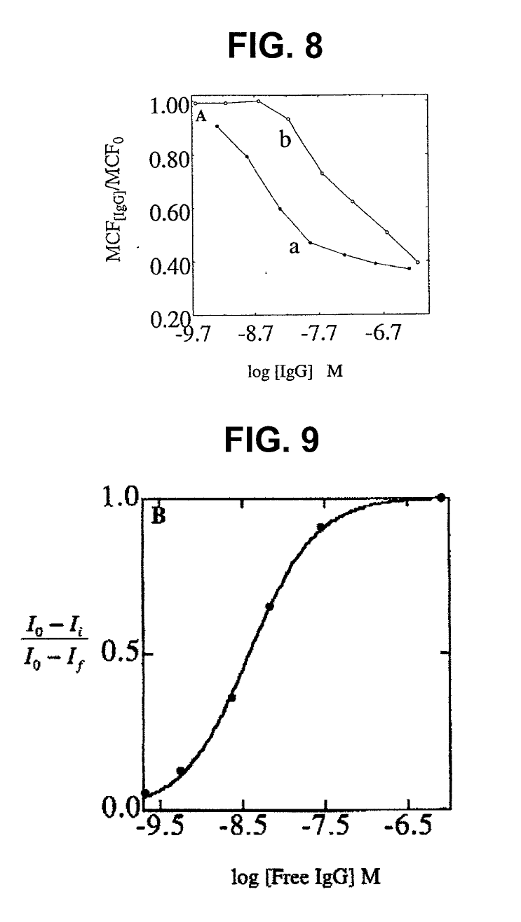

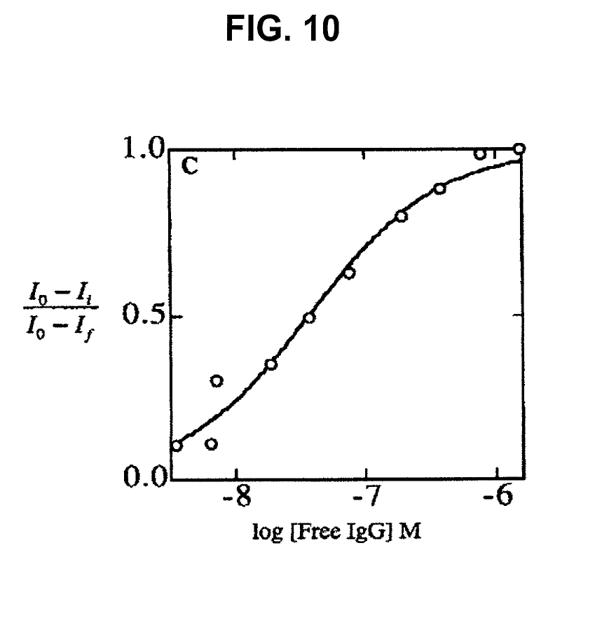

The equilibrium binding of TR-M1 to 5-FLAG on beads is shown in FIG. 8. Data shown is representative of three experiments. The line represented by letter “a” represents data collected in buffer with Ca²⁺ and line represented by letter “b” represents data collected in calcium-free buffer. FRET equilibrium binding of Texas-Red labeled M1 to 5-FLAG peptide-bearing beads in presence of Ca²⁺ and in Ca²⁺ free buffers is plotted. Subsequently, the data was analyzed as sigmoidal response curves shown in FIGS. 9 and 10.

FIG. 9 shows the sigmoidal analysis of the binding of TR-M1 to 5-FLAG bearing beads in presence of Ca²⁺ containing buffers. The normalized y-axis is as described in equation 2. The respective K_{d}s are determined to be ≈4.0 nM and 37.0 nM respectively.

FIG. 10 shows the sigmoidal analysis of the binding of TR-M1 to 5-FLAG bearing beads in Ca²⁺ free buffers. In calcium-free buffer TR-M1 binds to the beads albeit, at an affinity reduced by an order of magnitude (K_{d}≈37.0 nM) compared to when the cation is present (K_{d}≈4.0 nM).

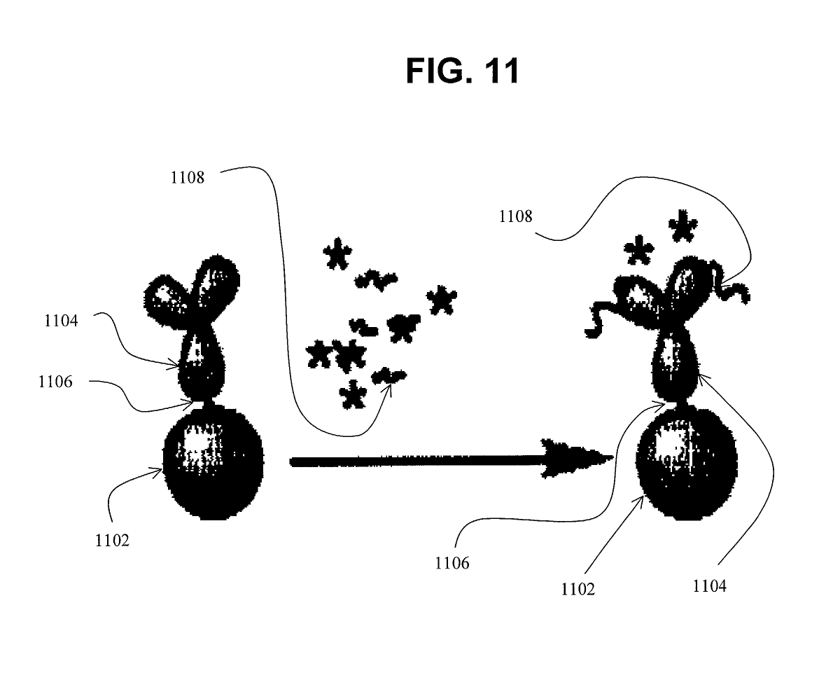

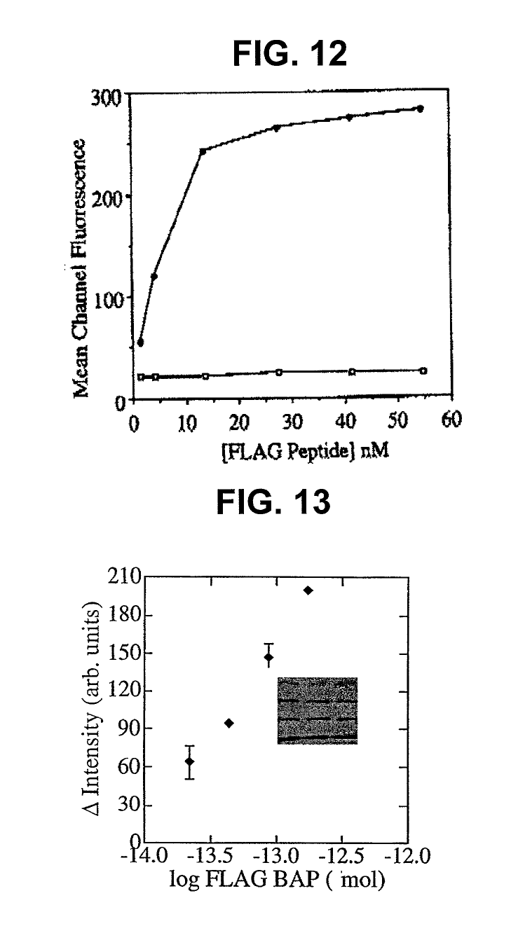

M2-bearing bead samples were incubated with increasing amounts of soluble 5-FLAG peptide. The resulting mixtures were centrifuged and resuspended in buffer, and then analyzed with the flow cytometer. The results of this analysis are shown in FIGS. 11 and 12.

FIG. 11 shows a schematic depiction of the capture of non-biotinylated fluorescent 5-FLAG peptide by biotinylated M2 IgG on beads. Bead 1102 has M2 IgG 1104 tethered on the surface via biotin 1106. Non biotinylated 5-FLAG peptide 1108 has a fluorescent tag conjugated to it. The biotinylated M2 IgG 1104 on beads 1102 captures the fluorescent non biotinylated 5-FLAG peptide. The resulting complex is detectable because of fluorescence present on the complexed 5-FLAG peptide.

FIG. 12 shows MCF of fluorescent FLAG peptide bound to M2 IgG on beads versus FLAG peptide. Solid circles represent M2 associated peptide, whereas open squares represent essentially background fluorescence from samples blocked with excess, dark FLAG peptide. The fluorescence intensities from the residual supernatants of the bead suspensions where analyzed by spectrofluorimetry. The intensity data (not shown) were used to generate a sigmoidal binding curve from which the monovalent K_{d} of the M2/FLAG interaction was determined to be ≈8.0 nM.

Applicability of the Present Invention to Detect Miniscule Amounts of Protein

The applicability of this methodology to assay development, by detecting femtomole amounts of N-Terminal FLAG bacteria alkaline phosphatase (FLAG BAP) fusion protein, in parallel with standard immunoprecipitation assays is shown in the present example. The characterizations performed here form the basis of generalizable assays for proteins with known epitopes.

Detection of FLAG BAP fusion Protein via Immunoblot and FRET on Beads

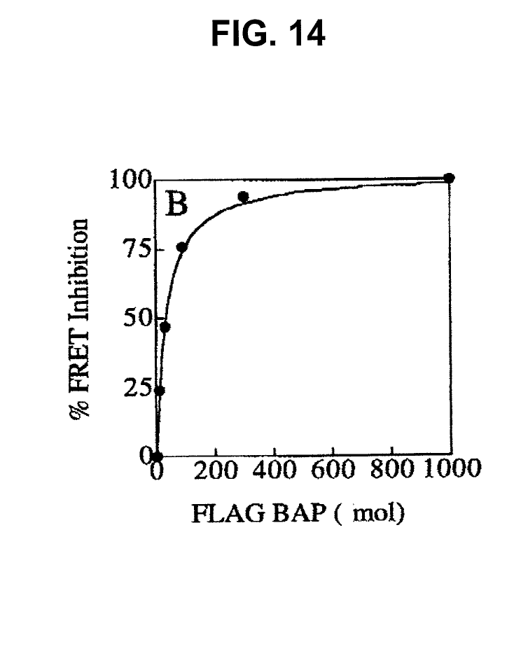

Standard immunoblot assays for the FLAG BAP were performed in parallel with the FRET assay on beads. The results are shown in FIGS. 13 and 14. The standard response curves for known amounts of FLAG BAP as determined by immunoblot are shown in FIG. 13. The inset in FIG. 13, displays the SDS-PAGE minigel of purified FLAG BAP protein. The increasing intensities of the triplicate bands correspond to the amount of added protein as plotted in the graph. The immunoblot data shown was conducted in triplicate.

The standard response curves for known amounts of FLAG BAP as determined by the beads are shown in FIG. 14. The y-axis in FIG. 14 corresponds to the inhibition in FRET. The increase in bead intensity as more TR-M1/FLAG BAP complexes are formed results in reduction of FRET. These results are representative of three separate experiments.

Various features of the FRET based determination are elaborated here. The magnitude of the FRET signal due to the binding of defined quantities of TR-M1 to a known number of biomolecule-bearing beads is shown in FIG. 8. From this data, a sigmoidal curve shown in FIG. 9 for the binding of TR-M1 to the beads was determined to have a K_{d}≈4.0. Thus aliquots of a concentration of TR-M1 for e.g. 10 nM, consistent with a FRET signal of a known range of 50% quenching, were premixed with a series of different [FLAG BAP] samples, and then incubated with FLAG peptide beads. The results are reported as the differences in intensity between the intensity of the control sample with zero [FLAG BAP] and the respective intensities of samples with TR-M1/FLAG BAP complexes. As shown in FIG. 14, the increasing amount of [FLAG BAP] results in an increase in the inhibition of FRET until a plateau is reached.

Data sets obtained from the standard response curves for known amounts of FLAG BAP as determined by immunoblot and the standard response curves for known amounts of FLAG BAP as determined by bead binding were fit to a simple binding curve using GraphPad Prism software. The correlation coefficient for the immunoblot results was 0.960, compared to that of the bead assay of 0.999. The results from the two methods are very well correlated. Both methods were able to detect femtomole quantities of the N-Terminal FLAG bacteria alkaline phosphatase referred to as FLAG BAP fusion protein. FLAG BAP is generally used to assure the functional integrity of anti-FLAG M1 and M2 monoclonal antibodies in immuno-detection, and immuno-purification applications.

The precision of the bead data, at both ends of the dynamic range, indicates that this method could potentially render more accurate determinations of unknowns than the immunoblot.

Therefore, the preferred method of the present invention utilizes the bead format. However, for some other embodiments, formats other than beads may be preferable. Furthermore, the applicability of this methodology to assay development is demonstrated by detecting femtomole amounts of biomolecule, such as fusion protein.

Criteria Important for Assay Development

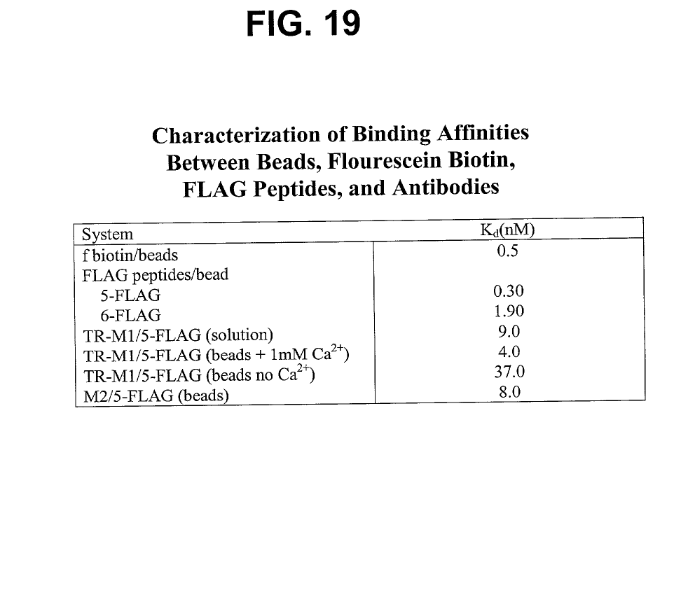

The below description is representative of one example of an approach to the development of potentially high throughput proteomic assays on beads. Such proteomic assays are described in the following reference: Borman, S. (2000) Proteomics: Taking Over Where Genomics Leaves Off Chem. & Eng. News 78, 31-37, the entire contents and disclosure of which hereby incorporated by reference. An important prerequisite to the development of such assays is the characterization of the components: interactions between beads and biotinylated ligand and between bead-borne (biotinylated) receptors and soluble ligands. The extent of such interactions, are represented by dissociation or affinity constants (K_{d}). The magnitude of affinity constants may be used to determine the viability of an assay. For example, tight binding (nM) is useful for molecular assemblies that are involved in the equilibrium capture of analytes, whereas moderate affinity constants (10-100 nM) may be appropriate for competitive displacement assays. A compiled set of K_{d} values determined from a series of equilibrium binding experiments with beads, peptides and antibodies, as shown in FIG. 19. Furthermore, the present invention seeks to demonstrate the applicability of this methodology to assay development, by detecting femtomole amounts of N-Terminal FLAG bacteria alkaline phosphatase (FLAG BAP) fusion protein, or other biomolecules.

FIG. 5 shows a general concept of the assays of the present invention. Such a general concept of the assay as described in the following reference: Tione Buranda, Gabriel Lopez, Peter Simons, Andrzej Pastuszyn and Larry Sklar, “Detection of Epitope-Tagged Proteins in Flow Cytometry: FRET Based Assays on Beads with Femto-mole Resolution” Analytical Biochemistry, Volume 298, No. 2, 2001 (in press), the entire contents and disclosure of which is hereby incorporated by reference. A general concept as described by the present invention may be that beads coated with the desired biomolecules are characterized to determine the site coverage of the biomolecules. Site coverage may be determined by measuring the fluorescence associated with the beads or from the analysis of residual fluorescence of the supernatants. Site coverage information obtained from the binding to a known number of beads suspended in a solution of known biomolecule concentration may be used to determine the affinity constant of the biomolecules to the beads. Panel A in FIG. 5 depicts the equilibrium binding of a defined quantity of 5-FLAG (K_{d} 0.3 nM) to a known number of beads. A site-occupancy of 10⁵ to 10⁶ peptides/bead is desired on a bead bearing 10 million streptavidin binding sites. The beads are then washed and re-suspended in buffer.

Another general concept as described by the present invention may be that fluorescently tagged biomolecules bind to other biomolecules. The fluorescence emission yield is sometimes quenched as a result of the interaction, thus it may be necessary to take steps to optimize the fluorescence intensity of surface bound biomolecules. In panel B in FIG. 5, native biotin is added to peptide-bearing beads to undo ostrich quenching, followed by washing to remove unbound biotin.

Another general concept as described by the present invention is that for an assay based on FRET, it may be necessary to know the magnitude of the signal that results from the binding to the energy donor-bearing biomolecules (on beads) of a known concentration of the biomolecules that bear the energy acceptor fluorescent tags. This type of characterization involves the determination of the equilibrium binding constant. The efficiency of FRET can be improved by maximizing the number of fluorescent tags on the biomolecules that bear the FRET acceptor tags. In panel C in FIG. 5, the binding of Texas red-labeled anti-FLAG antibodies to the FLAG peptides is manifested by the FRET quenching of the 520 nm emission of the fluorescein tag. The residual fluorescence intensity of quenched beads is on the order of 30% at antibody binding saturation.

Another general concept as described by the present invention is that in the absence of an analyte of interest, the magnitude of the FRET signal resulting from the binding of target biomolecules may be predetermined as described in panel C in FIG. 5. An unlabeled analyte or biomolecule that may bind to either the FRET donor-tagged or FRET acceptor-tagged biomolecule in the analyte solution may block the interaction of the tagged pair. This may result in a reduction of the FRET signal. Subsequent analysis of the inhibition of FRET may be determined to be proportional to the amount of analyte present in solution. Panel D in FIG. 5, depicts the basis of the FRET based detection scheme. Mixing of antibodies with FLAG tagged proteins (FLAG BAP) prior to incubation with beads leads to the inhibition of FRET due to the blocking of antibody binding sites. The inhibition of FRET is proportional to the concentration of the FLAG BAP protein.

Other data described in the present invention may be related to the FLAG system, but may be generalized for other biomolecules, as discussed above.

Characterization of Binding Biotinylated Ligands to Streptavidin Coated Beads

Although the binding of native biotin to streptavidin is of very high affinity (K≈10⁻M⁻¹), biotinylated molecules often bind to streptavidin with affinities that are reduced by several orders of magnitude. Such binding of native biotin to streptavidin is described in the following reference: Chilkoti, A., and Stayton, P. S. (1995) Molecular Origins of the Slow Streptavidin-Biotin Dissociation Kinetics J. Am. Chem. Soc. 117, 10622-10628, the entire contents and disclosure of which is hereby incorporated by reference. Such binding of biotinylated molecules to streptavidin is described in the following references: Buranda, T., Jones, G., Nolan, J., Keij, J., Lopez, G. P., and Sklar, L. A. (1999) Ligand Receptor Dynamics at Streptavidin Coated Particle Surfaces: A Flow Cyometric and Spectrofluorimetic Study J. Phys. Chem. B. 103, 3399-3410 and Wilchek M., and Bayer, E. A. (Eds.) (1990) Methods in Enzymology, Academic Press, Academic Press, London, the entire contents and disclosure of which is hereby incorporated by reference.

Thus, the determination of the binding capacity of biotinylated components to streptavidin-modified surfaces is an essential first step in the development of an assay. The binding affinity of biotinylated 5-FLAG to beads is on the order of 0.3 nM. Thus, in the absence of competing ligands of similar or higher affinity, the assembly of 5-FLAG peptides on beads can be achieved without significant loss of beadborne peptides over a period of days. Binding of fluorescent ligands to surfaces is typically associated with their quenching either due to contact with the protein (e.g. ostrich quenching) or self quenching as a function of site density on the bead surface. The total binding capacity of the beads was determined to be on the order of 10 million binding sites per bead using fluorescein biotin as a standard. This site coverage is ten times higher than previous lots of beads described elsewhere. Such site coverage is described in the following references: Buranda, T., Lopez, G. P., Keij, J., Harris, R., and Sklar, L. A. (1999) Peptides, Antibodies, and FRET on Beads in Flow Cytometry: A Model System Using Fluoresceinated and Biotinylated β-Endorphin Cytometry 37, 21-31 and Buranda, T., Jones, G., Nolan, J., Keij, J., Lopez, G. P., and Sklar, L. A. (1999) Ligand Receptor Dynamics at Streptavidin Coated Particle Surfaces: A Flow Cytometric and Spectrofluorimetic Study J. Phys. Chem. B. 103, 3399-3410, the entire contents and disclosure of which is hereby incorporated by reference. The FLAG peptides take up about seven million sites at maximum site coverage on the same beads. For an assay that depends on fluorescence intensity on beads steps must be taken to optimize the emission quantum yield of the bound peptides. Together, ostrich quenching and self-quenching can reduce the fluorescence intensity to less than 10% of the unquenched species. Self-quenching can be minimized by limiting the surface coverage to 100,000 ligands per bead. Defined coverage can be achieved because the affinity of the 5-FLAG peptide is known. The stoichiometry can be readily manipulated using a known number of beads with a defined surface coverage. Subsequently, biotin can be used to block the vacant sites, to eliminate ostrich quenching.

Characterization of the Binding of Antibodies to Peptide Bearing Beads

The binding of TR-M1 to the fluorescein-labeled 5-FLAG peptide may be shown by the quenching of peptide fluorescence, as shown in FIG. 8. The binding data was analyzed to determine the binding affinity of the antibody to the peptide in solution and on beads, as shown in FIGS. 9, 10 & 19. The sensitivity and dynamic range of the assay is defined by the affinity of the ligand/receptor pair. The efficiency of FRET in this system also plays a significant role in the sensitivity of the assay. At saturation, the quenching of peptide fluorescence is greater than 60%, as shown in FIG. 8 indicating that on the average the separation between the energy donor (fluorescein; D*) and acceptor (Texas Red; A) is less than Ro. This close proximity of the D*A pair allows for the sensitive detection of dilute amounts of TR-M1 bound to a small percentage of peptide sites on beads, or in solution. The monovalent affinity of the antibody/peptide interaction as determined in solution is about 9.0 nM, which is similar to the monovalent binding affinity (8.0 nM) of soluble FLAG peptide to M2 antibody on beads, as shown in FIGS. 11 & 12. The M2 data are provided to complete the characterization of the FLAG system.

Applicability of the Present Invention to Detect Miniscule Amounts of Protein

The applicability of this methodology to assay development, by detecting femtomole amounts of N-Terminal FLAG bacteria alkaline phosphatase (FLAG BAP) fusion protein, in parallel with standard immunoprecipitation assays is shown in the present example. The characterizations performed in this example may be used to form the basis of generalizable assays for proteins with known epitopes.

A standard procedure used to assay for proteins is immunoblotting. inmunoblots can detect proteins in biological fluids in the femtomole range and are capable of resolving different molecular weights. The bead-based assay discriminates between proteins on the basis of recognition, by the antibody to epitopes associated with specific proteins or due to specific tags on the protein. N-Terminal FLAG BAP is a standard used to assure the functional integrity of anti-FLAG M1 and M2 monoclonal antibodies in immuno-detection, and immuno-purification applications. The FLAG BAP is used here to demonstrate the applicability of the bead assay in the detection of a prototypical fusion tagged protein or a protein for which an antibody readily exists. The proof of concept experiment involves the assembly of 5-FLAG at known site densities on beads. Because the fluorescence of the beads corresponds to a known concentration of surface receptors, the subsequent changes define the amount of captured analytes, without signal interference from unbound analytes. Addition of a particular concentration of TR-M1, calibrated to give a defined FRET signal, as shown in FIG. 8, is added to the beads. In an assay format it is necessary to generate a standard curve, which can be used to determine the concentration of an unknown. Such a curve can be generated by pre-mixing fixed aliquots of TR-M1 (e.g. 10 nM) with a known protein (FLAG BAP) in concentrations ranging from about 0.1 to 2 orders of magnitude times the antibody concentration.

Standard immunoblot assays for the FLAG BAP were performed in parallel with the FRET assay on beads. The standard response curves for known amounts of FLAG BAP as determined by immunoblot are shown in FIG. 13. The inset in FIG. 13, displays the SDS-PAGE minigel of purified FLAG BAP protein. The increasing intensities of the triplicate bands correspond to the amount of added protein as plotted in the graph. The immunoblot data shown was conducted in triplicate. The standard response curves for known amounts of FLAG BAP as determined by the beads are shown in FIG. 14.

The binding of FLAG BAP to TR-M1 is then indicated as inhibition of FRET, as shown in FIG. 5 panel D, when the aliquots are mixed with fixed samples of 5-FLAG peptide bearing-beads. FIG. 7B displays the results of such an analysis, where the inhibition of FRET by known quantities of FLAG BAP is plotted as a function of concentration of the protein. Assaying for a FLAG tagged protein of indeterminate concentration, may then be achieved by the determination of the extent of FRET inhibition by the unknown protein relative to the points along the standard inhibition curve.

For purposes of the present invention, it may be important to point out that because the bead assay does not provide information on the molecular weight of the capture analyte, the specificity of the antibody and the presence of cross-reactive analytes may be a limiting factor. Thus interpretation of data may still require prior testing by immunoblotting to determine the presence of co-precipitates. Once developed for a specific biomolecule determination, the assay on beads has key advantages over immunoblotting: conservation of time, such as eight hours compared to an hour or less for the bead assay and, the possibility of sensitive and quantitative multiplex assays. Such advantages are described in the following references: Edwards, B. S., Kuckuck, F., and Sklar, L. A. (1999) Plug flow cytometry: An automated coupling device for rapid sequential flow cytometric sample analysis Cytometry; 37, 156-159, LundJohansen, F. Davis, K. Bishop, J. and Malefyt, R. D. (2000) Flow cytometric analysis of immunoprecipitates: High-throughput analysis of protein phosphorylation and protein-protein interactions Cytometry; 39, 250-259, Cai, H., White, P. S. Torney, D. Deshpande, A., Wang, Z. L., Marrone, B., and Nolan, J. P. (2000) Flow cytometry-based minisequencing: A new platform for high-throughput single-nucleotide polymorphism scoring Genomics; 66, 135-143, Choe, J. and vandenEngh, G. (2000) A novel fluorescent protein based sequencing vector for high throughput positive clone selection by flow cytometry Jounral of Investigative Medicine; 48, 86-86, Fluton, R. J., McDade, R. L., Kienker, L. J., and Kettman, J. R., (1997) Advanced Multiplexed Analysis with the Flowmetrix™ System Clinical Chemistry 43, 1749-1756, the entire contents and disclosure of which hereby incorporated by reference. The sensitivity of this assay may be controlled by the specificity and affinity of the antibody/biomolecule pair.

In an embodiment of the present invention, the present invention envisages to examine molecular assemblies using expressed proteins which involve FLAG tagged c-Myb proteins and FLAG-tagged ubiquitin-ligase proteins (28) from bacterial and insect cell lysates respectively. Such FLAG tagged c-Myb proteins are described in the following reference: Ness, S. A. (1996) The myb oncoprotein: Regulating a Regulator BBA Re. Cancer 1288, F123-F139, the entire contents and disclosure of which is hereby incorporated by reference. Such FLAG-tagged ubiquitin-ligase proteins are described in the following reference: Skowyra, D. Craig, K. L., Tyers, M., Elledge, S. J. and Harper, J. W. (1997) F-box proteins are receptors that recruit phosphorylated substrates to the SCF ubiquitin-ligase complex Cell; 91, 209-219, the entire contents and disclosure of which is hereby incorporated by reference.

Criteria for Construction of Microfluidic Columns

Bead Calibration

Binding data of the fluorescein biotin and FLAG peptides to streptavidin coated beads was obtained as described above. The magnitude of bound sites per bead were determined from centrifugation assays. From that analysis, the affinities of fluorescein biotin and the FLAG peptide were determined to be on the order of 0.5nM and 0.3nM respectively.

Preparation of Fluorescein Biotin Column

Streptavidin coated beads bearing ≈1×10⁶ fluorophores/bead were packed into the micro-channel. The analyte fluid at 3.0 mM biotin in 2 μL was added to the column and monitored as increasing emission intensity of the beads as the fluid flowed through the column.

Sample Delivery in Microchannels—The Laminar Flow Regime

The flow of analyte-fluid through the affinity micro-columns is laminar as is characterized by their low Reynolds numbers. The Reynolds number (Re) is a dimensionless parameter relating the ratio of inertial to viscous forces in a fluid. Laminar flow is typical for Re<1. For the current affinity micro-columns, the estimated Re is on the order of 10⁻⁵.

There are several limiting practical considerations for sample delivery on this scale: viscous forces dominate over inertial forces and with the virtual loss of turbulence diffusion is the basic method of mixing of soluble reagents. The friction between the transporting fluid and the interstitial surface of the affinity micro-column is manifested as a pressure drop (AP) across the microfluidic channel. In bead packed channels, the size of the beads and length of the column play an essential role in regulating the magnitude of ΔP. For the affinity micro-columns of dimensions similar to those in FIGS. 7A & 7B, empirical calculations have shown that, the pressure drop across an empty channel is ≈14 torr.

Passage of Soluble Analytes through Affinity Microcolumns—Fluorescein Biotin

The transport-limited kinetics and high affinity with a K_{d}≈10⁻¹³ M of the binding of biotin/streptavidin provides a method to characterize the fluid flow properties inside the channel. The volume of the reactor vessel is comprised of interstitial space between the receptor bearing beads. The average time for a molecule to diffuse across a distance d, is t=d²/2D where D is the diffusion coefficient of the molecule. In the column, d is small with a size in the range of 1 μm≧d. The time lapse for diffusive contact between the biotin and receptor surface is correspondingly small 0.1 sec for mAbs. Biotin is in large excess of the streptavidin receptors, the leading edge of the fluid passes through the column with negligible depletion of biotin. A direct correlation can therefore be made between the time-resolved increase in intensity and the velocity of the fluid. The flow rate through a column having ≈4.0 nL interstitial volume is on the order of 1.6 nLs⁻¹. Because the biotin experiment is essentially irreversible and quasi-unimolecular it serves as a useful calibration standard of the affinity micro-column, and facilitates the analysis of the more complex antibody binding data.

Some important factors related to the design, assessment and utility of affinity sensors are:

1. Beads derivatized with surface chemistry suitable for the attachment of fluorescently labeled biomolecules of interest are prepared and characterized in terms of functionality and receptor site densities by flow cytometry.

2. Second, calibrated beads are incorporated in microfluidic channels.

3. The resulting device that emerges replicates the basic elements of affinity chromatography with the advantages of (1) scale, (2) direct measurement of bound analyte on beads rather than the indirect determination from eluted sample typical of affinity chromatography, and (3) simultaneous detection of multiple analytes from columns comprised of discrete segments of diverse populations of receptor beads.

In conventional affinity chromatography, resolving the kinetics of ligand-ligate binding is indirect, based on the analysis of the elution profile and is dependent on retention times and peaks. The system described here has the advantage of direct and real time analysis and miniaturization.

In another embodiment of the present invention, it is possible to detect femtomole range of biomolecule 0.48 nM-4.8 nM. The high signal to noise ratio of these assays is due to the fact that the analytes are dark i.e. non fluorescent or do not contribute any background to the change in the fluorescence of the fluorescein tag. The assay has a wide dynamic range spanning nearly four orders of magnitude of analyte concentration. The good correlation between kinetic and equilibrium data enables one to determine concentrations of analytes from dynamic response, thus assays can be carried out in a few minutes, supplanting the need for time consuming steady state endpoint assays.

Fluorescent FLAG Peptide Bearing Beads

Several affinity micro-columns were prepared using ≈1.0×10⁶ peptides/bead. 2 μL plugs of Texas-Red labeled M1 anti-FLAG monoclonal antibodies (TR-M1) were eluted at varying concentrations in different affinity micro-columns. The binding of the antibody to the FLAG peptide was monitored as quenching of the peptide emission. To assure the specificity of the binding of TR-M1 mAbs to beads, samples of the mAbs were pre-equilibrated with varying amounts of a dark non-fluorescent peptide FLAG peptide and passed through the microcolumns.

Detection of Soluble Analytes in Affinity Micro-Columns

Detection of Native Biotin via Fluorescence Unquenching of Fluorescein Biotin

Under certain circumstances, binding of fluorescent ligands to streptavidin is characterized by the quenching of fluorescence of bound relative to free ligands. Typically, this type of quenching referred to as “ostrich quenching” occurs when the fluorophore (e.g., fluorescein) moiety of a biotinylated ligand associates with the receptor pocket adjacent to the biotin-moiety bearing site. Such a method is described in the following reference: Buranda, T., Jones, G., Nolan, J., Keij, J., Lopez, G. P., and Sklar, L. A. (1999) Ligand Receptor Dynamics at Streptavidin Coated Particle Surfaces: A Flow Cytometric and Spectrofluorimetic Study J. Phys. Chem. B. 103, 3399-3410, the entire contents and disclosure of which is hereby incorporated by reference.

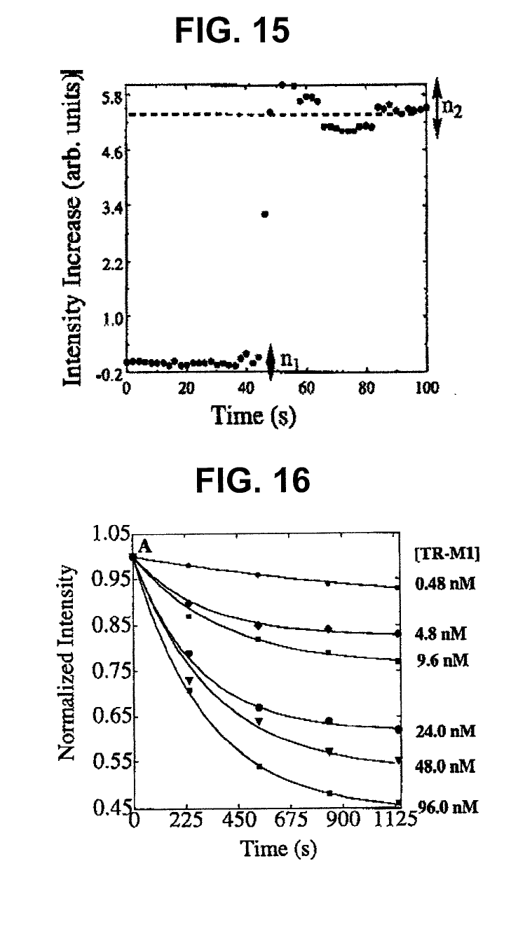

Ostrich quenching is dependent on the length and structure of the ligand. The Ostrich quenching interaction for fluorescein biotin is very weak with a K_{d}≈0.1 and readily obstructed by native biotin. Binding of fluorescein biotin to excess soluble streptavidin results in >90% quenching of the fluorescence. Addition of native biotin recovers the original intensity under diffusion-limited kinetics. The extent of quenching and recovery on beads depends on site occupancy of the fluorescein biotin. The data in FIG. 15 shows a fivefold increase in intensity of fluorescein biotin-bearing beads resulting from the injection of a 2 μL aliquot of 3 mM native biotin. The five-fold increase in intensity was consistent with a result from a flow cytometry measurement (data not shown). The data shows a good signal to noise ratio indicated by n₁. The magnitude of n₂ relative to n₁ is likely due to the disruption of the packing of beads upon initial contact with the plug of sample. It is likely that such disruption can be minimized through optimization of bead packing and sample injection procedures.

Real Time Detection of Anti-FLAG Monoclonal Antibodies via FRET

Biotinylated and fluorescein-tagged FLAG peptides were synthesized as described in the following reference: Tione Buranda, Gabriel Lopez, Peter Simons, Andrzej Pastuszyn and Larry Sklar, “Detection of Epitope-Tagged Proteins in Flow Cytometry: FRET Based Assays on Beads with Femto-mole Resolution” Analytical Biochemistry, Volume 298, No. 2, 2001 (in press), the entire contents and disclosure of which is hereby incorporated by reference. These biotinylated and fluorescein-tagged FLAG peptides were attached to streptavidin-coated beads. The density was ≈1×10⁶ peptides/bead in these preparations. These streptavidin coated beads carrying biotinylated and fluorescein tagged FLAG peptides were used for FRET analysis to determine the interaction of fluorescein labeled FLAG peptides with Texas-Red labeled anti-FLAG referred to as TR-M1 monoclonal antibodies (mAbs) by flow cytometry. Results from that study were compared to the analytical data collected in the affinity micro-columns described here. Several concentrations of TR-M1 mAbs were analyzed with affinity micro-columns. The results are described below. The binding of TR-M1 mAbs to FLAG peptide-bearing beads was monitored by FRET.

In FIG. 16, various concentrations of TR-M1 were injected into affinity columns at the rate of 1.6 nLs⁻¹. Data were normalized to the initial intensity of the beads before passage, through the column, of a 2 μL aliquot of TR-M1 mAbs. The data in FIG. 16 were fit to a kinetic model shown in Equation 1, expressed in terms of bimolecular interactions and diffusion-limited conditions.

$$\frac{ⅆ {Γ}_{AB}}{ⅆ t} = {k}_{f} {C}_{0} {Γ}_{A} - {k}_{b} {Γ}_{AB} = \frac{D}{δ} ( {C}^{b} - {C}_{0} ) \qquad ( 1 )$$

C^{b} and C₀ represent the concentrations of antibody in the bulk and at the liquid-solid interface respectively; Γ_{AB} is the surface concentration of FLAG peptides bound to antibodies; Γ_{A} is the surface concentration of unbound peptides; and k_{f} and k_{b} are the forward and reverse kinetic rate constants. D is the diffusion coefficient of the antibody and δ is the thickness of the steady-state diffusion-convection boundary layer established by fluid transport, assuming a linear gradient in concentrations (between C^{b} and C₀). The parameter D/δ represents the effects of diffusive transport of analytes to the surface receptors. The integral form of this equation is shown in Equation 2.

$$λθ - ( 1 + \frac{λ}{1 + κ} ) ln ( 1 - \frac{1 + κ}{κ} θ ) = \frac{1 + κ}{κ} λτ \qquad ( 2 )$$

In this equation θ=Γ_{AB}/Γ^{s} (where Γ^{s} (=Γ_{AB}+Γ_{A}) represents the total surface concentration of FLAG peptides. The adsorption constant,

$$κ = \frac{{k}_{f}}{{k}_{b}} {C}^{b} ,$$

the diffusion dependent rate of adsorption,

$$λ = \frac{{k}_{f} {δΓ}^{s}}{D} ,$$

and the dimensionless time normalized to diffusion time,

$$τ = \frac{t}{{t}_{d}} ,$$

(where the time that characterizes the diffusion process is

$${t}_{d} = \frac{{δΓ}^{s}}{D {C}^{b}} ) .$$

For a 6.2 μm diameter bead with 10⁶ receptors per bead, Γ^{S}=1.38×10⁻¹⁰ mol/dm².

Least squares error minimization between this equation and experimental data was performed with a Nelder-Mead Simplex algorithm. The fits to the experimental data yield the following parameter values: K_{d}=13.3±2.0 nM, k_{f}=(9.0±6.0)×10⁴ M⁻¹ s⁻¹, k_{b}=(1.2±0.8)×10⁻³ s⁻¹, D/δ=(1.0±0.9)×10⁻⁹ dm s⁻¹ where the errors are the standard deviations for the constants determined from the fittings of each experimental run. FIG. 3 shows the analysis of data corresponding to the endpoints in the egress of TR-M1 mAbs plugs through the affinity micro-columns. The equilibrium dissociation constant (K_{d}) from the binding curve is ≈10.0 nM,

Analysis of Anti-FLAG Monoclonal Antibodies

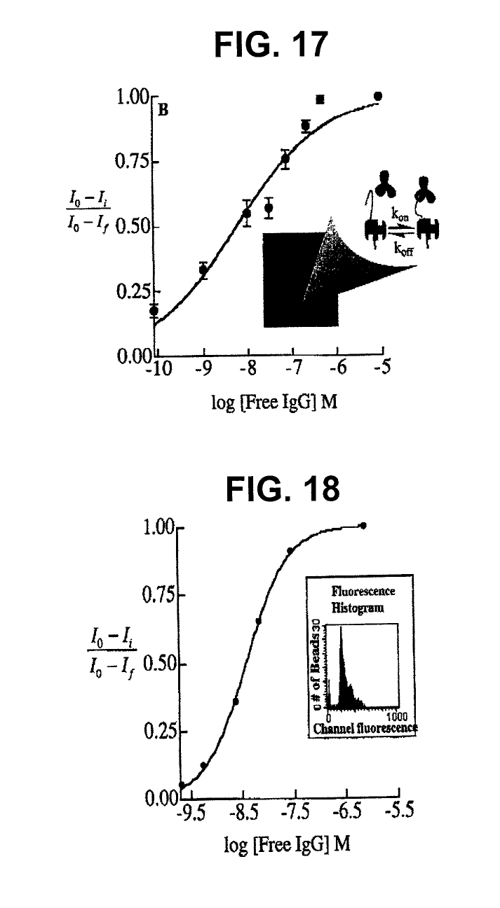

The determination of the kinetic and equilibrium binding constants between ligand/receptor systems is fundamental to the understanding of biological function as well as to the development of biomimetic systems. From a mechanistic approach, the temporal and spatial destiny of target analytes for e.g. TR-M1, traversing through the affinity micro-column might be described in terms of convective and diffusive transport, and reactive processes such as binding and dissociation. An additional point of detail in these analyses includes the generation of analyte concentration gradients in the transport fluid as well as those bound on beads. Thus, a complete study of such phenomena would be very complex. The formalism used to analyze the interaction of TR-M1 mAbs and biomolecules on beads reproduces the basic elements of mass transport dependent heterogeneous kinetics. However, it does not account for the putative concentration gradients of bound species that are likely to emerge during passage of the TR-M1 mAbs through the affinity micro-column. This simplifying assumption is possible because the micro-column is smaller than the spot size of the laser beam used to irradiate the beads. The FRET induced intensity changes associated with TR-M1/peptide complexes are integrated over the entire column thus, it is reasonable to make this simplified approach, with negligible loss of accuracy. The resulting binding and dissociation rate constants k_{f}=(9.0±6.0)×10⁴ M s⁻¹, k_{b}=(1.2±0.8)×10⁻³ s⁻¹ are in agreement with data reported in the literature on similar antibody-antigen interactions. A gratifying validation of this approach is shown, in the conservation of microscopic reversibility, by this close correlation of the affinity constant derived from kinetic data, K_{d}=k_{b}/k_{f}=13.3 nM and that derived from steady state data ≈10.0 nM shown in FIG. 17.

Analytical Characteristics of the Affinity Micro-column

In conventional affinity chromatography, resolving the kinetics of ligand-ligate binding is indirect, based on the analysis of the elution profile (retention times and peaks). The system described here has the advantage of direct and real time analysis and miniaturization. The high signal to noise ratio of these assays is due to the fact that the analytes are non-fluorescent (biotin) or do not contribute any background (TR-M1) to the change in the fluorescence of the fluorescein tag. The good correlation between kinetic and equilibrium data enables one to determine concentrations of analytes from dynamic response. Thus assays could potentially be carried out in a few minutes, supplanting the need for time consuming steady state endpoint assays. The close agreement between the binding constants determined from the kinetic and equilibrium data together with the flow cytometry data is a strong indicator of the reproducibility of these assays.

The analytical figures of merit (e.g. detection limit, dynamic range, sensitivity and precision) for the immunoreaction are derived in FIG. 17. The detection limit of the TR-M1 antibodies is in the sub-nanomolar range. Because of the tiny volumes allowed by the affinity micro-column, it is useful to refer to the detection limits in terms of the minimal detectible amount of TR-M1, which for the 2 μL aliquot is on the order of femtomoles. In general, the linear dynamic range of an immunoassay is considered to span 10 to 90% saturation of the antibody used (dashed horizontal lines in FIG. 17). This is usually equivalent to two orders of magnitude in analyte concentration (i.e. 0.1 K_{d}<analyte<10 K_{d}) in a conventional immunoassay such as the flow cytometry assay shown in FIG. 9. The data shown in FIG. 17, however indicates a linear dynamic range spanning 4 orders of magnitude. The dynamic range in FIG. 17 is extended on both the lower and upper limits in the concentration of the TR-M1 compared to FIG. 9. A variety of mechanisms could explain the wider dynamic range apparent in FIG. 17. A possible factor that might extend the dynamic range is the heterogeneity in the affinity of the antibody for the peptide sites throughout the column. The binding of the antibodies to beads in the flow cytometric analysis is largely characterized by monovalent binding. In the affinity column, the close packing of beads, can allow (higher affinity) bivalent binding of the antibody, while transport effects would tend to lower the monovalent affinity interactions. As a result, the dynamic range of the affinity micro-column is extended in both directions where, the aggregate K_{d} remains comparable to the true binding affinity. On the basis of FIG. 17, the sensitivity of the affinity column appears to be linear through out the dynamic range. The error bars shown in FIG. 17 are representative of a minimum of three measurements, taken per data point over an aggregate period of over a month for the complete set of data replicates. All data points and replicates were measured in distinct affinity columns.

FIG. 18 shows binding of TR-M1 mAbs to bead-borne FLAG peptides in flow cytometry in accordance with a method of the present invention. Binding of anti-FLAG mAbs to bead-borne FLAG peptides in flow cytometry is shown (K_{d}≈4.0 nM). Normalized intensities are derived from the means of fluorescence histograms (inset) of bead suspensions incubated with various concentrations of mAbs, and normalized to bead intensity prior to exposure to mAbs.

The specificity of the TR-M1/FLAG peptide system on beads is very high. Coating the beads with bovine serum albumin (BSA) during assay preparation appears to eliminate nonspecific interactions for the antibody concentration within the useful dynamic range of this assay. At very high concentrations of TR-M1, nonspecific interactions may emerge. When the soluble peptide is a thousand fold in excess of the TR-M1, negligible specific interaction is expected to occur with the bead borne peptides however, less than 10% of the FRET signal was observed. In a preliminary effort to establish the selectivity of this assay in a practical application, the detection of TR-M1 and the FRET-blocking non-fluorescent peptide may be achieved in a controlled manner in an analyte fluid comprised of blood serum and buffer.

The table in FIG. 19 shows the characterization of binding affinities between beads, flourescein biotin, FLAG peptides, and antibodies in accordance with a method of the present invention. The data from the binding affinities between f biotin/bead is from the sigmoidal analysis of binding measurements from centrifugation data. The affinity consents determined for f-biotin and 5-FLAG are similar in magnitude to the initial receptor concentration of (40,000 beads×10⁷ receptors/bead)/(6.023×10²³ receptor/mole×400 μL)≈0.17 nM. Due to the law of mass action considerations, the affinity constants may be limited by the initial receptor concentration thus could potentially be lower.

Although the present invention has been fully described in conjunction with the preferred embodiment thereof with reference to the accompanying drawings, it is to be understood that various changes and modifications may be apparent to those skilled in the art. Such changes and modifications are understood as included within the scope of the present invention as defined by the appended claims, unless they depart therefrom.

What is claimed is:

1. A sensing device comprising:
a vessel;
a plurality of sensor beads located within said vessel to form interstitial spaces therethrough; and
a plurality of biomolecules bound to at least a portion of said plurality of beads, each of said biomolecules having a fluorescent tag.

2. The sensing device of claim 1, wherein said vessel has a width of 250 μm to 500 μm.

3. The sensing device of claim 1, wherein said vessel has a length of 0.5 cm to 3.0 cm.

4. The sensing device of claim 1, wherein said vessel has a depth of 50 μm to 100 μm.

5. The sensing device of claim 1, wherein said plurality of beads are located in microfluidic channels in said vessel.

6. The sensing device of claim 5, wherein said microfluidic channels have a width of 10 μm to 500 μm.

7. The sensing device of claim 1, wherein said microfluidic channels are comprised of optically transparent material.

8. The sensing device of claim 7, wherein said optically transparent material comprises glass.

9. The sensing device of claim 7, wherein said optically transparent material comprises quartz.

10. The sensing device of claim 7, wherein said optically transparent material comprises a polymer.

11. The sensing device of claim 10, wherein said polymer comprises poly(dimethylsiloxane).

12. The sensing device of claim 1, wherein said plurality of sensor beads comprises at least two different types of sensor beads.

13. The sensing device of claim 1, wherein said plurality of biomolecules comprises at least two different kinds of biomolecules.

14. The sensing device of claim 13, wherein each of said two different kinds of biomolecules includes a different fluorescent tag.

15. The sensing device of claim 14, wherein said sensing device comprises at least two sensing regions, each of said sensing regions including one of said at least two different kinds of biomolecules.

16. The sensing device of claim 15, wherein said vessel includes obstructive features therein for preventing flow of said sensor beads between said at least two sensing regions.

17. The sensing device of claim 13, wherein said plurality of beads comprise at least two different kinds of beads and each of said different kinds of biomolecules are bound to a respective type of said at least two different types of sensor beads.

18. The sensing device of claim 1, further comprising spacer beads within said vessel.

19. The sensing device of claim 1, wherein said sensing device further comprising foundation beads within said vessel.

20. The sensing device of claim 19, wherein said foundation beads are comprised of glass or a metallic.

21. The sensing device of claim 20, wherein said foundation beads have a diameter of 30 μm to 1000 μm.

22. The sensing device of claim 1, wherein said vessel includes obstructive features therein for preventing said sensor beads from flowing along said vessel.

23. The sensing device of claim 22, wherein neighboring obstructive features of said obstructive features are located 5 μm to 20 μm from each other.

24. The sensing device of claim 1, wherein said sensor beads are 1 μm to 1000 μm in diameter.

25. The sensing device of claim 1, wherein said sensor beads are coated with at least one coating of said plurality of biomolecules.

26. The sensing device of claim 25, wherein bound biomolecules of said plurality of biomolecules are bound to said plurality of bead by biotin.

27. The sensing device of claim 1, wherein said interstititial spaces each has a volume of 1 nL to 1000 nL.

28. A method for detecting the binding of two biomolecules comprising the following steps:
providing a plurality of first biomolecules, each of said first biomolecules having a first fluorescent tag, each of said first biomolecules being bound to a respective substrate of a plurality of substrates;
providing a plurality of second biomolecules, each of said second biomolecules having a second fluorescent tag;
binding at least portion of said second biomolecules to at least a portion of said first biomolecules to form complexes, wherein said plurality of first biomolecules and said plurality of second biomolecules prior to said binding step have a pre-complexing total fluorescence and wherein said complexes and free second biomolecules after said binding step have a post-complexing total fluorescence; and
detecting any difference between said pre-complexing total fluorescence and said post-complexing total fluorescence.

29. The method of claim 28, wherein each said plurality of substrates comprises beads.

30. The method of claim 29, wherein said beads comprise poly(dimethylsiloxane).

31. The method of claim 29, wherein said beads have a diameter of 0.1 μm to 1000 μm.

32. The method of claim 28, wherein said first biomolecules comprise at least two different types of first biomolecules.

33. The method of claim 28, wherein said plurality of substrates comprises at least two different types of beads, and each of said at least two different types of first biomolecules are bound to a respective one of said at least two different types of beads.

34. The method of claim 28, wherein said second biomolecules comprises at least two different types of second biomolecules.

35. The method of claim 28, wherein said second fluorescent tag comprises at least two different fluorescent tags bound to a respective one type of said at least two different types of second biomolecules.

36. The method of claim 28, wherein said first biomolecules are capable of detecting an epitope of said second biomolecules.

37. The method of claim 36, wherein said epitope comprises SEQ ID NO: 1.

38. The method of claim 36, wherein said binding step takes place in the presence of calcium ions.

39. The method of claim 28, wherein energy is transferred between said first fluorescent tag and said second fluorescent tag, wherein said first fluorescent tag and said second fluorescent tag are at a distance of 10 μm to 200 μm from each other.

40. The method of claim 28, wherein at least one member selected from the group consisting of said first fluorescent tag and said second fluorescent tag comprises a donor molecule having an emission spectrum and the other member of said group comprises an acceptor molecule having an absorption spectrum, and wherein said emission spectrum and said absorption spectrum overlap.

41. The method of claim 28, wherein the detection is done using a flow cytometer.

42. The method of claim 28, wherein the detection is done using a spectrophotometer.

43. The method of claim 28, wherein the detection is done using a microscope.

44. The method of claim 28, wherein, any difference between said pre-complexing total fluorescence and said post-complexing total fluorescence is detected using fluorescence resonance energy transfer.

45. The method of claim 28, further comprising the step of determining the amount of said second biomolecule based on the detected difference between said pre-complexing total fluorescence and said post-complexing total fluorescence.

46. The method of claim 28, wherein said pre-complexing total fluorescence and said post-complexing total fluorescence is measured using fluorescence resonance energy transfer.

47. A sensing device comprising:
a suspension of a plurality of sensor beads; and
a plurality of biomolecules bound to at least a portion of said plurality of beads, each of said biomolecules having a fluorescent tag.

48. The sensing device of claim 47, wherein said plurality of sensor beads comprises at least two different types of sensor beads.

49. The sensing device of claim 47, wherein said plurality of biomolecules comprises at least two different kinds of biomolecules.

50. The sensing device of claim 49, wherein each of said two different kinds of biomolecules includes a different fluorescent tag.

51. The sensing device of claim 47, wherein said sensor beads are 1 μm to 1000 μm in diameter.
